(19) United States
(12) Patent Application Publication
(10) Pub. No.: US 2009/0165172 A1
(43) Pub. Date: Jun. 25, 2009

(54) METHODS AND COMPOSITIONS FOR INCREASING BIOMASS IN GENETICALLY MODIFIED PERENNIALS USED FOR BIOFUELS

(75) Inventors: Albert P. Kausch, Stonington, CT (US); Melvin Oliver, Columbia, MO (US)

Correspondence Address: GAUTHIER & CONNORS, LLP, 225 FRANKLIN STREET, SUITE 2300, BOSTON, MA, 02110, US

(73) Assignee: BOARD OF GOVERNORS FOR HIGHER EDUCATION, STATE OF RHODE ISLAND AND PROVIDENCE, PROVIDENCE, RI (US)

(21) Appl. No.: 12/274,778

(22) Filed: Nov. 20, 2008

Related U.S. Application Data
(63) Continuation of application No. PCT/US07/69651, filed on May 24, 2007.
(60) Provisional application No. 60/808,074, filed on May 24, 2006.

(51) Int. Cl. C12N 15/82; A01H 1/02; A01H 5/00

(52) U.S. Cl. 800/279; 800/278; 800/260; 800/298; 800/289; 800/290; 800/284

(57) ABSTRACT

Genes can be introduced into plants that confer desirable traits such as, drought and stress tolerance, insect and pest resistance, as well as traits for enhancing biofuel production, such as increased vegetative biomass and prolonged vegetative growth. The development of reproductive structures diverts resources from vegetative growth resulting in lower biomass and fixed growing seasons. Disclosed herein are methods and compositions for generating controlled vegetative growth and prolonged growing seasons for the purpose of increasing biomass in plants used for biofuels.

### PRIORITY INFORMATION

This application claims priority to International Patent Application No. PCT/U.S.07/69651 filed on May 24, 2007 which claims priority to Provisional Application No. 60/808,074 which was filed on May 24, 2006, which is incorporated by reference in its entirety herein.

### BACKGROUND OF THE INVENTION

1. Field of the Invention

The present disclosure relates to plant genome modification methods that result in asexual (flowerless) or floral deficient phenotypes and increases in vegetative biomass and growing season.

2. Description of the Prior Art

The improvement of many plants, such as those used for the production of biofuels, through conventional breeding commonly relies on the identification of a single improved trait within a cultivar and is restricted to germplasm that is capable of sexual crosses to yield fertile offspring. An improved trait within a given cultivar once identified, is followed by extensive back-crossing, election and evaluation to produce a commercially viable product. This process can require up to fifteen years, and is restricted to traits confined to the gene pool of the plant.

In contrast, many important crop plants can be genetically transformed with genes from other species, even across kingdom barriers. The introduction of cloned genes into plant cells and recovery of stable fertile transgenic plants can be used to make modifications in a plant, and has created the potential for genetic engineering of plants for crop improvement. Genetic modifications by plant transformation allow stable alterations in biochemical processes that direct traits such as increased yield, disease and pest resistance, increased vegetative biomass, herbicide tolerance, nutritional quality, drought and stress tolerance, as well horticultural qualities such as pigmentation and growth, and other agronomic characteristics for crop improvement. In these methods, foreign DNA is introduced into the eukaryotic plant cell, followed by isolation of cells containing the foreign DNA integrated into the cell's DNA, to produce stably transformed plant cells.

The utilization of energy crops produced on American farms as a source of renewable fuels is a concept with great relevance to current ecological and economic issues on both national and global scales. Development of a significant national capacity to utilize perennial forage crops, such as switchgrass Panicum virgatum, L., Poaceae), as biofuels could provide independence from foreign oil, a cleaner source of energy for road fuel to diminish greenhouse gas emissions, benefit our agricultural economy by providing an important new source of income for farmers, and allow for more productive use of land currently within the Conservation Reserve Program (CRP). In addition energy production from perennial cropping systems, which are compatible with conventional farming practices, would help reduce degradation of agricultural soils, lower national dependence on foreign oil supplies, and reduce emissions of greenhouse gases and toxic pollutants to the atmosphere.

One drawback that arises regarding transgenic improvement of perennials, such as switchgrass and other plant based biofuels, is the biological fact that when plants produce flowers, carbon resources are allocated to floral development at the expense of vegetative biomass. As an open pollinated species, switchgrass expresses tremendous genetic diversity, with wide variations in its basic chromosome number (2n=18), typically ranging from tetraploid to octoploid. Morphologically switchgrass in its southern range can grow to more than 3 m in height, but what is most distinctive is the deep, vigorous root system, which may extend to depths of more than 3.5 m. It reproduces both by seeds and vegetatively and, with its perennial life form, a stand can last indefinitely once established. Standing biomass in root systems may exceed that found aboveground, giving perennial grasses such as switchgrass, an advantage in water and nutrient acquisition even under stressful growing conditions.

Physiologically, switchgrass, like maize, is a C4 species, fixing carbon by multiple metabolic pathways with high water use efficiency. In general C4 plants such as grasses will produce 30% more biomass per unit of water than C3 species such as trees and broadleaved crops and grasses and are well adapted to the more arid production areas of the mid-western US where growth is more limited by moisture supply.

To date switchgrass has been bred primarily to enhance its nutritional value as a forage crop for livestock. Thus, it has been managed primarily is a hay crop for which high leaf to stem ratio and high nutrient content are important. These targets are quite different from the criteria for biofuels crops for which high biomass yield, high cellulose, and low ash content are important for high energy conversion and low contamination of combustion systems.

The life cycle of flowering plants in general can be divided into three growth phases: vegetative, inflorescence, and floral. In the vegetative phase the shoot apical meristem (SAM) generates leaves that later will ensure the resources necessary to produce fertile offspring. Upon receiving the appropriate environmental and developmental signals the plant switches to floral, or reproductive, growth and the SAM enters the inflorescence phase (I1) and gives rise to an inflorescence with flower primordia. During this phase the fate of the SAM and the secondary shoots that arise in the axils of the leaves is determined by a set of meristem identity genes, some of which prevent and some of which promote the development of floral meristems. Once established, the plant enters the late inflorescence phase (I2) where the floral organs reproduced. Two basic types of inflorescences have been identified in plants: determinate and indeterminate. In determinate species the SAM eventually produces floral organs and the production of meristems is terminated with a flower. The SAM of indeterminate species is not converted to a floral identity and will therefore only produce floral meristems from its periphery, resulting in a continuous growth pattern. The regulation of meristem identity and plant architecture has been investigated in a number of dicotyledonous plants including Arabidopsis, Antirrhinum, tomato, and tobacco. The molecular mechanisms controlling floral development have also been investigated in some important monocotyledonous seed crops such as rice, maize and forage grasses. Genetic and molecular studies with two dicot plants, Antirrhinum and Arabidopsis, have shown that the genetic network controlling flower development is conserved in the two dicot species. After the transition from vegetative to reproductive development, floral meristems are initiated by the action of a set of genes called floral meristem identity genes. Among them, FLORICAULA (FLO) of Antirrhinum and its Arabidopsis counterpart LEAFY (LFY) seem to play the most important role in the reproductive transition to establish floral fate. FLO/LFY encode putative transcription factors that do not show significant homology to any known genes. In strong flo and lfy mutant plants, flowers are transformed into inflorescence shoots demonstrating a role in the transition from vegetative to floral meristem mutant phynotypes of FLO/LFY homologs in several other dicot species suggest that the function of FLO/LFY during reproductive development is largely conserved among the dicots, though its function during other stages of development may vary. The fact that FLO/LFY expression precedes that of other meristem-identity genes with flower-specific expression, and because flo/lfy loss-of-function mutations have the strongest effect on meristem identity suggests that FLO/LFY are responsible for the initial steps in flower initiation.

At the time when wild-type plants begin to produce flowers, flo/lfy mutants continue to produce leaves and associated lateral shoots. In monocots, the FLO/LFF homologs have also been identified in, rice, ryegrass and maize and zfl2, the mutants of two duplicate FLORICAULA/LEAFY homologs in maize (Zea mays L. ssp. mays), a monocot species with dramatically different flower and inflorescence morphology from that of dicot species, exhibit a disruption of floral organ identity and patterning, as well as defects in inflorescence architecture and in the vegetative to reproductive phase transition, demonstrating that these genes share conserved roles with their dicot counterparts in flower and inflorescence patterning. In addition, the FLORICAULA/LEAFY homologs from different species have a high similarity in amino acid sequences, and are highly conserved in the C-terminal regions. These important characteristics of the FLORICAULA/LEAFY-like genes in plants provide the basis for induction of meristem modifications used for increasing vegetative biomass.

In one example, if the expression of the FLORICAULA/LEAFY-like genes in genetically modified switchgrass are turned off, the transgenic plants grown in the field will maintain total vegetative growth, unable to produce flowers, and produce increased amounts of vegetative growth. Coincidentally, the vegetatively grown transgenic plants should also eliminate any potential risks of transgene escape through a reproductive pathway. Since the FLORICAULA/LEAFY homologs from different species have high homology in amino acid sequences, especially in the C-terminal regions, part of the FLORICAULA/LEAFY homologs can be easily isolated from switchgrass through PCR. This strategy has been successfully used to isolate the FLO/LFY homologs in other species. Preliminary results using this strategy have resulted in amplification of a DNA fragment with predicted size from a turfgrass (data not shown). In order to turn off the FLO/LFY-like genes in switchgrass, both antisense and RNAi approaches have been used to achieve the goal of down-regulation resulting in floral sterility and increased biomass. These two approaches have been applied in a number of plant species to down-regulate the expression of various genes by a knock-down strategy to decrease the transcript levels of their targeted endogenous counterparts, resulting in the loss-of-function mutations. In other examples, the FLORICAULA/LEAFY-like gene promoters can be isolated used to direct the specific ablation of floral meristem development, by directing the expression of a cytotoxic protein or a cell growth inhibitor, to generate the desired increased biomass production.

One consideration in applying a meristem fate strategy for increased biomass production is whether the engineered total vegetative growth can be controlled at will, i.e., the FLO/LFY-like (or other meristem fate control) genes in switchgrass can be kept functioning normally in transgenic plants during seed multiplication and then turned off for total vegetative growth when grown under the non-controlled field conditions. Such regulation of expression of foreign gene in plants has been achieved with promoters responsive to environmental stimuli or synthetic chemicals. However, this method of gene regulation, when applied in large scale, is difficult to do either because there is not a system that can guarantee a very tight control for gene expression, or because there are no inducing agents, such as chemicals, that can be easily and safely used in the field without causing other problems. In this scenario, site-specific DNA recombination provides a solution.

Site-specific recombination is a process involving reciprocal exchange between specific DNA sites (referred to as target sites) catalyzed by specialized proteins site-specific recombinases. As such, these recombinases can alter genomic DNA sequences in specific ways providing powerful tools for the development of a new generation of molecular technology for crop improvement. Site-specific recombinases recognize specific DNA sequences, and in the presence of two such recombination sites they catalyze the recombination of DNA strands. In these site-specific recombination systems, recombinases can catalyze excision or inversion of a DNA fragment according to the orientation of their specific target sites. Recombination between directly oriented sites leads to excision of the DNA between them, whereas recombination between inverted target sites causes inversion of the DNA between them. Some site-specific recombination systems do not require additional factors for their function and are capable of functioning accurately and efficiently in various heterologous organisms. The initial work on site-specific recombination in plant cells was conducted using the Cre/lox system of bacteriophage P1. In 1990, it was demonstrated that Cre recombinase could excise, invert, or integrate extrachromosomal DNA molecules in tobacco protoplasts. In the same year, other crucial evidence was provided that the cre gene could be stably expressed in plant cells (tobacco) without any evident deleterious effects on plant development, and that the Cre protein could recognize and recombine lox sites integrated into the plant genomic DNA. It was also demonstrated that the successful passage of the cre gene from one plant to another through cross-pollination. It was also indicated that the Cre protein did not consistently support complete excisions, indicating the need for further development of this system. The idea of using the Cre/lox system to remove selectable markers was presented and extended to Arabidopsis plants. Expected results were obtained and confirmed earlier observations that the efficiency of recombinations strongly depends on Cre expression levels. The formation of extensive leaf phenotypic sectoring, especially pronounced in cross-breeding experiments, was a major manifestation of such aberrant action of Cre recombinase in plant cells. Nevertheless, the feasibility of the Cre/lox system for genetic engineering of plant genomes has been proven and well documented in additional papers.

The FLP/FRT recombination system functioning endogenously in eukaryotic yeast cells was identified as another particularly attractive candidate for catalyzing efficient recombination reactions in heterologous eukaryotic cells used a modified FLP-coding sequence from pOG44 to synthesize a chimeric plant FLP gene driven by the maize ubiquitin promoter to show activity of FLP recombinase in maize and rice cells.

In 1994, an encouraging report was published on FLP-mediated activation of a hygromycin-resistance gene in the tobacco genome by cross-pollination. Soon after, experiments performed demonstrated that FLP/FRT system activity could be controlled in a precise manner in maize cells with high molecular fidelity. In an attempt to explore further applications of FLP recombinase in plant cells, the feasibility of using FLP recombinase in Arabidopsis for making plant hybrids was recently investigated. The researchers first obtained two transgenic lines of Arabidopsis highly expressing a modified FLP gene, or an FRT-containing recombination-reporter construct.

By cross-pollination between individuals from these two transgenic lines, the in planta functionality of FLP/FRT system in Arabidopsis for excisional recombination (FIG. 2) was successfully demonstrated. The FLP-expressing plant was then crossed with a transgenic line expressing a FRT-flanked male sterility gene; the FLP successfully excised the male-sterility-causing-elements flanked by FRT sites, producing fertile hybrid plants (FIG. 3). To extend this application to cereal 20 crops for hybrid production, specifically to rice, the same researchers have studied the in planta efficacy of FLP-mediated site-specific DNA recombination in rice. They hypothesized that rice plants containing a construct in which the rice ubiquitin promoter and the reporter gusA coding region is separated by the hygromycin (hyg) gene flanked by directly oriented FRT sites will not show GUS activity. In addition, when crossed to a plant expressing the FLP recombinase, FLP should excise the blocking fragment (hyg gene) thus bringing together the ubiquitin promoter and the downstream gusA gene, giving rise to GUS expression in the hybrid plant. Using Agrobacterium-mediated transformation, Luo et al, obtained transgenic plants with the FLP-containing construct or the FRT-containing recombination-reporter construct. Transient assays of the FLP recombinase activity in planta using leaves from the transgenic rice seedlings demonstrate the in 30 vivo functionality of FLP/FRT system in transgenic rice plants (FIG. 4). After cross-pollination between the FLP-expressing planets and FRT-containing plants, hybrid seeds were harvested and planted to produce F₁ plants. Leaves were then sampled from the hybrid seedlings and stained for GUS activity. While the majority of the hybrid progeny exhibited a uniform GUS expression, the progeny of selfed parental rice plants did not showed detectable GUS activity (FIG. 4). Molecular analysis further confirmed the FLP-mediated site-specific DNA excision (data not shown). This observation clearly demonstrates the efficient operation of FLP recombinase in catalyzing excisional DNA recombination, indicating that the FLP/FRT recombination system functions in rice.

Recent work on the application of site-specific recombination system for plant genome modification has extended the use of the FLP/FRT system to turfgrass (FIG. 5) and cotton cells (FIG. 6). The experimental data obtained has demonstrated that the FLP/FRT recombination system can unction efficiently in plant species, such as the grasses, and that site-specific recombination systems will operate as a powerful tool for plant genome engineering. Based on all the data on the FLP/FRT site-specific recombination system can control, through hybridization to FLP-expressing plants, the down-regulation of a flower-specific gene (FLORICAULA/LEAFY homolog) in switchgrass, producing controlled vegetative growth of transgenic plants.

This system can be used to produce and control the expression of total floral sterility in plants resulting in continuous vegetative growth and an increase their biomass.

### SUMMARY OF THE INVENTION

In contrast, many important crop plants can be genetically transformed with genes from other species, even across kingdom barriers. The introduction of cloned genes into plant cells and recovery of stable fertile transgenic plants can be used to make modifications in a plant, and has created the potential for genetic engineering of plants for crop improvement. Genetic modifications by plant transformation allow stable alterations in biochemical processes that direct traits such as increased yield, disease and pest resistance, increased vegetative biomass, herbicide tolerance, nutritional quality, drought and stress tolerance, as well horticultural qualities such as pigmentation and growth, and other agronomic characteristics for crop improvement. In these methods, foreign DNA is introduced into the eukaryotic plant cell, followed by isolation of cells containing the foreign DNA integrated into the cell's DNA, to produce stably transformed plant cells.

The utilization of energy crops produced on American farms as a source of renewable fuels is a concept with great relevance to current ecological and economic issues on both national and global scales. Development of a significant national capacity to utilize perennial forage crops, such as switchgrass (Panicum virgatum, L., Poaceae), as biofuels could provide independence from foreign oil, a cleaner source of energy for road fuel to diminish greenhouse gas emissions, benefit our agricultural economy by providing an important new source of income for farmers, and allow for more productive use of land currently within the Conservation Reserve Program (CRP). In addition energy production from perennial cropping systems, which are compatible with conventional farming practices, would help reduce degradation of agricultural soils, lower national dependence on foreign oil supplies, and reduce emissions of greenhouse gases and toxic pollutants to the atmosphere.

One drawback that arises regarding transgenic improvement of perennials, such as switchgrass and other plant based biofuels, is the biological fact that when plants produce flowers, carbon resources are allocated to floral development at the expense of vegetative biomass. As an open pollinated species, switchgrass expresses tremendous genetic diversity, with wide variations in its basic chromosome number (2n=18), typically ranging from tetraploid to octoploid. Morphologically switchgrass in its southern range can grow to more than 3 m in height, but what is most distinctive is the deep, vigorous root system, which may extend to depths of more than 3.5 m. It reproduces both by seeds and vegetatively and, with its perennial life form, a stand can last indefinitely once established. Standing biomass in root systems may exceed that found aboveground, giving perennial grasses such as switchgrass, an advantage in water and nutrient acquisition even under stressful growing conditions.

Physiologically, switchgrass, like maize, is a C4 species, fixing carbon by multiple metabolic pathways with high water use efficiency. In general C4 plants such as grasses will produce 30% more biomass per unit of water than C3 species such as trees and broadleaved crops and grasses and are well adapted to the more arid production areas of the mid-western US where growth is more limited by moisture supply.

To date switchgrass has been bred primarily to enhance its nutritional value as a forage crop for livestock. Thus, it has been managed primarily as a hay crop for which high leaf to stem ratio and high nutrient content are important. These targets are quite different from the criteria for biofuels crops for which high biomass yield, high cellulose, and low ash content are important for high energy conversion arid low contamination of combustion systems.

A method has been developed for the controlled total vegetative growth of switchgrass and other perennial plants, using down-regulation of a plant gene that determines reproductive transition together with a site-specific DNA recombination system, as a strategy for selective floral ablation resulting in continuous vegetative growth and increased biomass. The implementation of controllable total vegetative growth in genetically modified transgenic switchgrass will not only increase biomass production but will eliminate any and all potential risks of transgene flow, allowing the necessary gene stacking requirements for further genetic modification. A similar strategy can also be applied to other plant species when developing genetically engineered products using recombinant DNA technology, for example, to those that can be propagated vegetatively and used for production of biofuels.

A method for the controlled total vegetative growth of switchgrass and other perennial plants has been developed, using down-regulation of a plant gene that determines reproductive transition together with a site-specific DNA recombination system, as a strategy for selective floral ablation resulting in continuous vegetative growth and increased biomass. The implementation of controllable total vegetative growth in genetically modified transgenic switchgrass will not only increase biomass production but will eliminate any and all potential risks of transgene flow, allowing the necessary gene stacking requirements for further genetic modification. A similar strategy can also be applied to other plant species when developing genetically engineered products using recombinant DNA technology, for example, to those that an be propagated vegetatively and used for production of biofuels.

The ability to increase biomass in plants used for biofuels will increase the efficiency of producing the derived final products such as ethanol, hydrogen or biodiesel. Therefore, methods are needed that will maximize resources for biofuel production. Disclosed herein are methods for generating controlled total vegetative growth in plants, such as asexual or floral deficient plants, which will prolong and increase vegetative growth. In addition, the controlled total vegetative growth in transgenic plants will be generated as the product of two parental lines to allow for improvement by marker assisted breeding and transgenic gene stacking strategies. Further advantages are that controlled total vegetative growth in perennial plants like switchgrass, will: 1) not produce or produce insignificant numbers of viable seeds or pollen, thus preventing or decreasing the potential risk of transgene escape into the surrounding environment by outcrossing with wild or nontransgenic plants; 2) allow for germplasm control, for commercialization and the development of genetic lines that are specifically selected for agronomic-traits to enhance yield and other characteristics; 3) allow for additional stacked traits to be incorporated into the genome.

Methods to generate controlled total vegetative growth in perennial plants, such as asexual or male and/or female floral deficient plants, are disclosed herein by functionally deleting floral structures, such as the floral apex. In one example, a cytotoxic molecule, such as a barnase gene or an anti-sense floral-specific gene, is driven by a floral-specific promoter creating either no or defective floral structures, such as flowerless or archegonia and/or antheridia deficient plants. For example, expression of the cytotoxin, that in one example produces a protein product that decreases the presence and/or production of tapetum, which results in antheridial deficiencies. In another example down regulation of a gene involved in the development of the floral apex or directing the ablation of floral meristems will result in asexual (flowerless) or floral deficiency phenotypes and prolonged vegetative growth.

Also disclosed herein is the use of site-specific recombination to generate male and female sterile perennial plants. The hybrid seeds produced from such plants will produce asexual plants, thus resulting in plants with increased vegetative growth. In addition, second-generation asexual perennials will allow for advanced breeding techniques to be applied to the parents thus, protecting the proprietary lines developed by seed companies. In one example, the method includes crossing a first fertile plant having one or more desirable traits, with a second fertile plant, which can also have one or more desirable traits. For example, the first plant can be resistant to glufosinate and the second plant resistant to glyphosate. The first fertile plant contains a vector which includes a floral-specific promoter operably linked to a blocking sequence, such as a selectable marker, wherein the blocking sequence is flanked by recombining site sequences. The vector also includes a cytotoxic sequence downstream of the promoter and selectable marker, and positioned such that its expression is activated by the floral-specific promoter in the presence of a recombinase, which results in recombination at the recombining site sequences and removal of the blocking sequence. The second fertile plant includes another vector which includes a promoter, such as a constitutively active or inducible promoter, operably linked to a recombinase. If an inducible promoter is used, the second fertile plant is contacted with an inducing agent, before, during, or after crossing the first and second fertile plants. The constitutively active promoter, or inducing agent that activates the inducible promoter, permits recombinase expression. The expressed recombinase protein interacts with the recombining sites of the other vector, resulting in recombination, removal of the blocking sequence such that the floral-specific promoter is now operably linked to the cytotoxin, thereby driving expression of the cytotoxin. The resulting progeny of such a cross have an asexual or floral deficient phenotype.

In an alternative example, instead of using two vectors, all of the elements can be placed on a single vector, which is transfected into plants or plant cells. For example, the first or the second fertile plant contains a vector which includes a floral-specific promoter operably linked to a blocking sequence, wherein the blocking sequence is flanked by a recombining site sequence, a cytotoxic sequence downstream of the blocking sequence such that the cytotoxic sequence is operably linked to the promoter upon site-specific re-combination, and a promoter (such as a constitutively active or inducible promoter) operably linked to a recombinase. If an inducible promoter is used, the plant transfected with the vector is contacted with an inducing agent, before, during, or after crossing the plants. The inducing agent, or constitutively active promoter, promotes recombinase expression. The expressed recombinase protein interacts with the recombining sites, resulting in recombination, removal of the blocking sequence such that the floral-specific promoter is now operably linked to the cytotoxin, thereby driving expression of the cytotoxin. The resulting progeny of such a cross are asexual or floral deficient thus achieving an in increased vegetative growth. A method to produce total vegetative growth by engineering down-regulated expression of a plant gene determining reproductive transition from a vegetative meristem has been developed. This down regulation can be controlled using a site-specific DNA recombination system to facilitate seed production. This strategy provides a solution to increasing vegetative biomass of switchgrass but also can be used in other plant species that can be propagated vegetatively, or plants, such as vegetables, for which seeds are not the final targeted products.

These and other features and objectives of the present invention will now be described in greater detail with reference to the accompanying drawings, wherein:

### BRIEF DESCRIPTION OF THE FIGURES

FIG. 1 is a schematic diagram of plasmids that can be used in an Flp/Frt recombinase system. The FRT plasmid includes a promoter, FRT and FRTm sequences that flank a blocking sequence (such as the bar selectable marker) followed by a cytotoxic sequence. The FLP plasmid includes a selectable marker (such as bar) and a promoter that drives expression of FLP. Expression of FLP causes recombination at the FRT sites, excising the blocking sequence in the FRT plasmid, allowing the promoter to drive expression of the cytotoxin sequence;

FIG. 2 is an example of histochemical staining of GUS activity;

FIGS. 3A-C. show FLP-mediated recombination for use in hybrid plant production;

FIGS. 4A-C illustrate FLP-mediated site-specific DNA including after cross pollination between FLP-expressing plants and FRT-containing plants; and

FIGS. 5-8 are schematic diagrams of methods that can be used to generate sterile plants using a recombinase system.

### SEQUENCE LISTING

The nucleic acid sequences listed in the accompanying sequence listing are shown using standard letter abbreviations for nucleotide bases. Only one strand of each nucleic acid sequence is shown, but the complementary strand is understood as included by any reference to the displayed strand.

SEQ ID NO: 1 is a rice tapetum-specific (rts) sequence.

| # SEQUENCE LISTING | |
|---|---|
| (1) | GENERAL INFORMATION: |
| (iii) | NUMBER OF SEQUENCES: 4 |
| (2) | INFORMATION FOR SEQ ID NO: 1: |
| (i) | SEQUENCE CHARACTERISTICS: #pairs |
| (A) | LENGTH: 1753 base |
| (B) | TYPE: nucleic acid |
| (C) | STRANDEDNESS: double |
| (D) | TOPOLOGY: linear |
| (ii) | MOLECULE TYPE: DNA (genomic) |
| (iii) | HYPOTHETICAL: NO |
| (iv) | ANTI-SENSE: NO |
| (vi) | ORIGINAL SOURCE: |
| (A) | ORGANISM: Oryza sat - #iva |
| (B) | STRAIN: IR54 |
| (vii) | IMMEDIATE SOURCE: |
| (B) | CLONE: RTS2 |
| (xi) | SEQUENCE DESCRIPTION: SEQ ID NO: 1: |
| | GAGCTCACCG GCGAGGCGGT GCGTCTCCTC GGAGATGTGG TAGAAGCTGG CG |
| #CCCCATTT 60 | CATGGCGGCG AGCGGGCCGG GCGGCCTCCT GCCGCAGCGG GATTCGGTGG CT |
| #AGACCGAT 120 | CTGTGGGTGG AGGACGGGGA CGAGGTAGAG GAGACAGAGG CGGCATTGGA AG |
| #AGGGGAAG 180 | AGGAGGAGGA AGTGGTGGCA GGCAGAGGCG GATGAGGAAC TTGCGCCAGC GA |
| #CGTGGATA 240 | TGGAGGGGGC GACGGCAATG GGGAGGCGGC GATGGAAGCG AGGAGATGGG CA |
| #GGCGGCGG 300 | AGGCAGCGGT GGATTTTTTT TTTCTTTTTC TTTTTCGGAC CCTTTACCCT GC |
| #TCGGTGAT 360 | TCTTCTTTTT TATACAGCAC GACGGCTTCT CCTATTCACG ACGCCTCGGC TG |
| #GACCATGG 420 | ACCGTTGGCC ACTGGAGCAT TCTTCCATGA TCTAGATTTT TTTTTTCACT CA |
| #ACTTTACT 480 | ACTTCACATC TGATGGCTGG TGTTGAATTC ATTGTGCATC CAACGGTCAT TA |
| #TTAAATTG 540 | ATGACGTGGC GCAATGAGGT GACGAAACAC TTTACTTTTT TTACTACTTT AG |
| #ATCTGTCG 600 | GCAGGAGTCC CAGATAGATA CTTGAGCTGG TTAGTTGGGT TTTGGATGGA GT |
| #AACTTTCT 660 | GCAGACTGCA ACATTCTGAC ACACGTAGCA GCACAAAAGA GTTGCGAACA AA |
| #CTTGGACT 720 | GTTAACATGT CAACGCATAA AACTGAAAAA AAAAACCTGT CAAAATGCAT AA |
| #TAAATAAA 780 | ACTGAAAAAA AATAAGAATA AATGTTGAGA GTGGGATTTG AACCCACGCC CT |
| #TTCGGACC 840 | AGAACCTTAA TCTGGCGCCT TAGACCAACT CGGCCATCTC AACTTTTTGC TC |
| #TGTCATCC 900 | AAACAAAGTT ATAAGAAATC ATATAATAAT AACTAAGACT TGATGCCTCA GT |
| #AGTTTAGT 960 | TAAACTAATT TGAATTTGTT AGTACAGTTT GCATTTCAAA TTGTTCCAAT TT |
| #GGACGCCA 1020 | CGGCTGGTTT CAGTTGCTCA CGACGCCTCA CACACATATT TTGCTTCCTT GC |
| #TTGTGACA 1080 | CTAGGGCACA AAACTCCAAC ACTCAAACGA CACTTCACGC ATCTCTCCTG AA |
| #ATCTTGCA 1140 | CCCCCCAACT CTGCATCGTC GCGTATAAAA TGCAGACCAA ACCCCAGCTC AA |
| #CTCTGCAT 1200 | CATCATCATC AACTCGATAG AAAAAGAAAG AAATTAAAAA GAAAATCACG GC |
| #GCGTGAGC 1260 | TTGCAGAGAC AGCAATGGTG AGAGTTGCTG CCGCCGCGGC GGTGCTCGTG CT |
| #GGCGGCGG 1320 | CGGCGGCGGC GGCGGCGGCC ATGGCCGCCG AGCCGCCCAC CGATGACGGC GC |
| #GGTCCGGG 1380 | TGGCGGCGGG GCTGACGAAG TGCGTGTCCG GGTGCGGTAG CAAGGTGACC TC |
| #CTGCTTGC 1440 | TCGGCTGCTA CGGCGGCGGC GGCGGCGCCG CCGCCGCCGC GACGGCGATG CC |
| #GTTCTGCG 1500 | TCATCGGCTG CACCAGCGAC GTCTTGTCCT GCGCCACCGG CTGCTCCACC TC |
| #GCTCTGAT 1560 | TAAGTACTAA TGAAGTAATT AACCGCGCTA ATTAATAATA AATCGCACCT AC |
| #GTATGCAC 1620 | ATGTGGACTC GCTTGACTAA TTAAATACTG CCATGCGAAT GCGATTAGTG GA |
| #TTATGAAA 1680 | AGAGGAAATG TAAGAACTCA TGGCTCTCTC TGTGCCATGC CTGTACTGCA TT |
| #GAAATGAA 1740 | #1769 AACT GATATACAA |

SEQ ID NO: 2 is a nucleic acid sequence of a rice tapetum-specific promoter.

| | |
|---|---|
| (2) | INFORMATION FOR SEQ ID NO: 2: |
| (i) | SEQUENCE CHARACTERISTICS: #pairs |
| (A) | LENGTH: 1262 base |
| (B) | TYPE: nucleic acid |
| (C) | STRANDEDNESS: single |
| (D) | TOPOLOGY: linear |
| (ii) | MOLECULE TYPE: DNA (genomic) |
| (iii) | HYPOTHETICAL: NO |
| (iv) | ANTI-SENSE: NO |
| (xi) | SEQUENCE DESCRIPTION: SEQ ID NO: 2: |
| | GAGCTCACCG GCGAGGCGGT GCGTCTCCTC GGAGATGTGG TAGAAGCTGG CG |
| #CCCCATTT 60 | CATGGCGGCG AGCGGGCCGG GCGGCCTCCT GCCGCAGCGG GATTCGGTGG CT |
| #AGACCGAT 120 | CTGTGGGTGG AGGACGGGGA CGAGGTAGTG GAGACAGAGG CGGCATTGGA AG |
| #AGGGGAAG 180 | AGGAGGAGGA AGTGGTGGCA GGCAGAGGCG GATGAGGAAC TTGCGCCAGC GA |
| #CGTGGATA 240 | TGGAGGGGGC GACGGCAATG GGGAGGCGGC GATGGAAGCG AGGAGATGGG CA |
| #GGCGGCGG 300 | AGGCAGCGGT GGATTTTTTT TTTCTTTTTC TTTTTCGGAC CCTTTCACCT GC |
| #TCGGTGAT 360 | TCTTCTTTTT TATACAGCAC GACGGCTTCT CCTATTCACG ACGCCTCGGC TG |
| #GACCATGG 420 | ACCGTTGGCC ACTGGAGCAT TCTTCCATGA TCTAGATTTT TTTTTTCACT CA |
| #ACTTTACT 480 | ACTTCACATC TGATGGCTGG TGTTGAATTC ATTGTGCATC CAACGGTCAT TA |
| #TTAAATTG 540 | ATGACGTGGC GCAATGAGGT GACGAAACAC TTTACTTTTT TTACTACTTT AG |
| #ATCTGTCG 600 | GCAGGAGTCC CAGATATGTA TACTTGAGCT GGATTAGTTG GGTTTTGGAT GG |
| #AGTAACTT 660 | TCTGCAGACT GCAACATTCT GACACACGTA GCAGCACAAA AGAGTTGCGA AC |
| #AAACTTGG 720 | ACTGTTAACA TGTCAACGCA TAAAACTGAA AAAAAAAACC TGTCAAAATG CA |
| #TAATAAAT 780 | AAAACTGAAA AAAAATAAGA ATAAATGTTG AGAGTGGGAT TTGAACCCAC GC |
| #CCTTTCGG 840 | ACCAGAACCT TAATCTGGCG CCTTAGACCA ACTCGGCCAT CTCAACTTTT TG |
| #CTCTGTCA 900 | TCCAAACAAA GTTATAAGAA ATCATATAAT AATAACTAAG ACTTGATGCC TC |
| #AGTAGTTT 960 | AGTTAAACTA ATTTGAATTT GTTAGTACAG TTTGCATTTC AAATTGTTCC AA |
| #TTTGGACG 1020 | CCACGGCTGG TTTCAGTTGC TCACGACGCC TCACACACAT ATTTTGCTTC CT |
| #TGCTTGTG 1080 | ACACTAGGGC ACAAAACTCC AACACTCAAA CGACACTTCA CGCATCTCTC CT |
| #GAAATCTT 1140 | GCACCCCCCA ACTCTGCATC GTCGCGTATA AAATGCAGAC CAAACCCCAG CT |
| #CAACTCTG 1200 | CATCATCATC ATCAACTCGA TAGAAAAAGA AAGAAATTAA AAAGAAAATC AC |
| #GGCGCGTG 1260 | #1278 AA |

SEQ ID NOS: 3 and 4 are primers used to obtain a bamase coding sequence.

| | |
|---|---|
| 5′-CACAGGAAACAGGATCCGCGG-3′ | (SEQ ID NO: 3) |
| and | |
| 5′CGCGAGCTCGCCGGAAAGTGAAATTGACC-3′ | (SEQ ID NO: 4) |

### DETAILED DESCRIPTION OF THE INVENTION

### Abbreviations and Terms

The following explanations of terms and methods are provided to better describe the present disclosure and to guide those of ordinary skill in the art in the practice of the present disclosure. As used herein, the singular forms “a” or “an” or “the” include plural references unless the context clearly dictates otherwise. For example, reference to “a transgenic plant” includes one or a plurality of such plants, and reference to “the floral-specific promoter” includes reference to one or more floral-specific promoters or their homologues and equivalents thereof known to those skilled in the art, and so forth.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

Anther-specific gene: A gene sequence that is primarily expressed in the anther, relative to expression in other plant tissues. Includes any anther-specific gene whose malfunction or functional deletion results in male-sterility. Examples include, but are not limited to: anther-specific gene from tobacco (GenBank Accession Nos. AF376772-AF376774), and Osg4B and Osg6B (GenBank Accession Nos. D21159 and 21160).

Anther-specific promoter: A DNA sequence that directs a higher level of transcription of an associated gene in anther tissue relative to the other tissues of the plant. Examples include, but are not limited to: anther-specific gene promoter from tobacco (GenBank Accession Nos. AF376772-AF376774), and the promoters of Osg4B and Osg6B (GenBank Accession Nos. D21159 and D21160).

Antisense molecules: Nucleic acid molecules that are specifically hybridizable or specifically complementary to either RNA or the plus strand of DNA. In a cell, the antisense nucleic acids hybridize to the corresponding mRNA, forming a double-stranded molecule. The antisense nucleic acids interfere with the translation of the mRNA, since the cell will not translate a mRNA that is double stranded. In one example, an antisense oligomer is about 15 nucleotides. The use of antisense molecules to inhibit the in vitro translation of genes is well known in the art.

An effective floral-specific antisense molecule, such as a tapetum-specific antisense molecule, is characterized by its ability to decrease or inhibit the expression of the floral-specific molecule. Complete inhibition is not necessary for effectiveness, some sequences are capable of inhibiting the expression of a floral-specific molecule by at least 15%, 30%, 40%, 50%, 60%, 70%, 80%, 90%, 95%, 98%, 99% or 100%. An effective antisense molecule is additionally characterized by being sufficiently complementary to a floral-specific encoding nucleic acid sequences. Sufficient complementary indicates that the effective antisense molecule can specifically disrupt the expression of a floral-specific gene, and not significantly alter the expression of genes other than a floral-specific gene.

Asexual: A plant lacking floral structures such that it is incapable of participating either as a male or female parent in sexual reproduction and propagates vegetatively.

Barnase: A cytotoxic extracellular ribonuclease. Examples of particular barnase DNA and protein sequences that can be used to practice the methods disclosed herein can be found on Genbank (for example Accession Nos: S01373, X15545 and M14442). Includes sequences obtained from Bacillus amyloliquefaciens, as well as other organisms, such as Aspergillus oryzae (RNase-T1). Includes variants of wild-type barnase sequences that retain barnase biological function, such as cytotoxic activity.

Binding/stable binding: A oligonucleotide sequence, such as an antisense sequence, binds or stably binds to a target nucleic acid if a sufficient amount of the oligonucleotide forms base pairs or is hybridized to its target nucleic acid, to permit detection of that binding. Binding can be detected by physical or functional properties of the target:oligonucleotide complex. Binding between a target 15 and an oligonucleotide can be detected by any method known to one skilled in the art, including functional and physical binding assays. Binding can be detected functionally by determining whether binding has an observable effect upon a biosynthetic process such as expression of a gene, DNA replication, transcription and translation.

Physical methods of detecting the binding of complementary strands of DNA or RNA are well known in the art, and include such methods as DNase I or chemical footprinting, gel shift and affinity cleavage assays, Northern blotting, dot blotting and light absorption detection procedures. For example, a method that is widely used involves observing a change in light absorption of a solution containing an oligonucleotide (or an analog) and a target nucleic acid at 220 to 300 nm as the temperature is slowly increased. If the oligonucleotide or analog has bound to its target, there is a sudden increase in absorption at a characteristic temperature as the oligonucleotide (or analog) and target dissociate or melt. The binding between an oligomer and its target nucleic acid is frequently characterized by the temperature (Tₘ), at which 50% of the oligomer is melted from its target. A higher Tₘ means a stronger or more stable complex relative to a complex with a lower Tₘ.

Blocking sequence: A DNA sequence of any length that blocks a promoter from effecting expression of a targeted gene. In one example, a vector of the present disclosure includes a floral-specific promoter operably linked to a cytotoxin, the promoter and cytotoxin being separated by a blocking sequence that is in turn bounded on either side by recombining site sequences. In the absence of the appropriate recombinase, the cytotoxin is not expressed. Presence of the appropriate recombinase effects the removal of the blocking sequence at the specific recombining site sequences, thereby directly linking the cytotoxin and the floral-specific promoter, allowing expression of the cytotoxin by the floral-specific promoter. Examples of blocking sequences include, but are not limited to non-coding DNA sequences, and selectable marker gene sequences.

Comprises: A term that means “including.” For example, “comprising A or B” means including A or B, or both A and B, unless clearly indicated otherwise.

Cytotoxin: An agent, such as a protein, that can kill a cell, or decrease the ability of the cell to function as it would in the absence of the toxin. For example, expression of a cytotoxic gene, or antisense molecule, can be fatal to the cell expressing such a gene or molecule. Examples include, but are not limited to ribonuclease genes, such as barnase (from Bacillus amylstiquefaciens) and RNase-T1 (from Aspergillus oryzae) and antisense molecules that decrease or inhibit cell development or kill cells, such as molecules that interfere with tapetum development. Additional examples include avidin, auxin production-related genes, DAM methylase, and Diphtheria toxin. Decrease in or prevention of transgene escape: A substantial reduction in the viability of pollen, which decreases the risk of a transgene in a transgenic plant escaping into another plant individual or population. Complete prevention of transgene escape is not necessary for effectiveness. In one example, a substantial reduction in pollen viability is when no more than 0.1% of the pollen produced by a plant is viable (as compared to the viability of wild-type pollen of the same variety of plant), for example, no more than about 0.01% of the pollen is viable, for example, no more than about 0.001% of the pollen is viable, or even less.

Deletion: The removal of a sequence of a nucleic acid, for example DNA, the regions on either side being joined together.

Desirable trait: A characteristic which is beneficial to a plant, such as a commercially desirable, agronomically important trait. Examples include, but are not limited to: resistance to insects and other pests and disease-causing agents (such as viral, bacterial, fungal, and nematode agents); tolerance or resistance to herbicides; enhanced stability; increased yield or shelf-life; environmental tolerances (such as tolerance to drought, heat, chilling, freezing, excessive moisture, salt stress, or oxidative stress); male sterility; and nutritional enhancements (such as starch quantity and quality; oil quantity and quality; protein quality and quantity; amino acid composition; and the like). On one example, a desirable trait is selected for through conventional breeding. In another example, a desirable trait is obtained by transfecting the plant with a transgene(s) encoding one or more genes that confer the desirable trait to the plant.

Floral deficient: A plant that is lacking, or is functionally deficient in, one or several parts of the male or female structures contained within a single flower or inflorescence effectively resulting in either male or female sterility.

Floral-specific gene: gene sequence that is primarily expressed in floral tissue or during the transition from a vegetative to floral meristem, such as the tapetum, anther, ovule, style, or stigma, relative to the other tissues of the plant. Includes any floral-specific gene whose malfunction or functional deletion results in sterility of the plant either directly or by preventing fertilization of gametes through floral deficiencies.

Floral-specific promoter: A DNA sequence that directs a higher level of transcription of an associated gene in floral tissues or during the transition from vegetative to floral meristem relative to the other tissues of the plant. Examples include, but are not limited to: meristem transition-specific promoters, floral meristem-specific promoters, anther-specific promoters, pollen-specific promoters, tapetum-specific promoters, ovule-specific promoters, megasporocyte-specific promoters, megasporangium-specific promoter-0, integument-specific promoters, stigma-specific promoters, and style-specific promoters. In one example, floral-specific promoters include an embryo-specific promoter or a late embryo-specific promoter, such as the late embryo specific promoter of DNH 1 or the HVA1 promoter, the GLB1 promoter from corn, and any of the Zein promoters (Z27). In another example, floral-specific promoters include the FLO/LFY promoter from switchgrass.

The determination of whether a sequence operates to confer floral specific expression in a particular system (taking into account the plant species into which the construct is being introduced, the level of expression required, etc.), is preformed using known methods, such as operably linking the promoter to a marker gene (e.g. GUS, and GFP), introducing such constructs into plants and then determining the level of expression of the marker gene in floral and other plant tissues. Sub-regions which confer only or predominantly floral expression, are considered to contain the necessary elements to confer floral specific expression.

Functional deletion: A gene is functionally deleted when the function of the gene or gene product is reduced or eliminated. For example, anti-sense molecules can be used to functionally delete a gene. In another example, a cell or tissue is functionally deleted when the function of the cell or tissue is reduced or eliminated. For example, cytotoxic genes, such as barnase, can be used to functionally delete floral-specific cells, such as the tapetum, thereby resulting in sterility of the plant.

Functionally equivalent: Nucleic acid sequence alterations in a vector that yield the same results described herein. Such sequence alterations can include, but are not limited to, conservative substitutions, deletions, mutations, frameshifts, and insertions. For example, in a nucleic acid including a barnase sequence that is cytotoxic, a functionally equivalent barnase sequence may differ from the exact barnase sequences disclosed herein, but maintains its cytotoxic activity. Methods for determining such activity are disclosed herein.

Isolated: An “isolated” biological component (such as a nucleic acid or protein) has been substantially separated, produced apart from, or purified away from other biological components in the cell of the organism in which the component naturally occurs, i.e., other chromosomal and extrachromosomal DNA and RNA, and proteins. Nucleic acids and proteins that have been “isolated” include nucleic acids and proteins purified by standard purification methods. The term also embraces nucleic acids and proteins prepared by recombinant expression in a host cell as well as chemically synthesized nucleic acids, proteins and peptides.

Nucleic acid: A deoxyfibonucleotide or ribonucleotide polymer in either single or double stranded form, and unless otherwise limited, encompasses known analogues of natural nucleotides that hybridize to nucleic acids in a manner similar to naturally occurring nucleotides.

Oligonucleotide: A linear polynucleotide (such as DNA or RNA) sequence of at least 9 to 35 nucleotides, for example at least 15, 18, 24, 25, 27, 30, 50, 100 or even 200 nucleotides long.

ORF (open reading frame): A series of nucleotide triplets (codons) coding for amino acids without any termination codons. These sequences are usually translatable into a peptide.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

Peptide: A chain of amino acids of which is at least 4 amino acids in length. In one example, a peptide is from about 4 to about 30 amino acids in length, for example about 8 to about 25 amino acids in length, such as from about 9 to about 15 amino acids in length, for example about 9-10 amino acids in length.

Perennial: A plant which grows to floral maturity for three seasons or more. Whereas annual plants sprout from seeds, grow, flower, set seed and senesce in one growing season, perennial plants persist for several growing seasons. The persistent seasonal flowering of perennial plants may also, but not necessarily, include light and temperature requirements that result in vernalization. Examples include, but are not limited to: certain grasses, such as turfgrass, forage grass or ornamental grasses; trees, such as fruit and nut crop trees (for example bananas and papayas), forest and ornamental trees, rubber plants, and shrubs; grapes; roses; and wild rice.

Pollen-specific gene: A DNA sequence that directs a higher level of transcription of an associated gene in microspores and/or pollen (i.e., after meiosis) relative to the other tissues of the plant. Examples include, but are not limited to: pollen-specific promoters LAT52, LAT56, and LAT59 from tomato (GenBank Accession Nos. BG642507, X56487 and X56488), rice pollen specific gene promoter PSI (GenBank Accession No. Z16402), and pollen specific promoter from corn (GenBank Accession No. BD136635 and BD136636).

Pollen-specific promoter: A gene sequence that is primarily expressed in pollen relative to the other cells of the plant. Includes any pollen-specific gene whose malfunction or functional deletion results in male-sterility. Examples include, but are not limited to: LAT52, LAT56, and LAT59 from tomato (GenBank Accession Nos. BG642507, X56487 and X56488), PSI (GenBank Accession No. Z16402), and pollen specific gene from corn (GenBank Accession No. BD136635 and BD136636).

Promoter: An array of nucleic acid control sequences that directs transcription of a nucleic acid. A promoter includes necessary nucleic acid sequences near the start site of transcription, such as, in the case of a polymerase II type promoter, a TATA element. A promoter also optionally includes distal enhancer or repressor elements that can be located as much as several thousand base pairs from the start site of transcription. Both constitutive and inducible promoters are included.

Specific, non-limiting examples of promoters that can be used to practice the disclosed methods include, but are not limited to, a floral-specific promoter, constitutive promoters, as well as inducible promoters for example a heat shock promoter, a glucocorticoid promoter, and a chemically inducible promoter. Promoters produced by recombinant DNA or synthetic techniques may also be used. A polynucleotide encoding a protein can be inserted into an expression vector that contains a promoter sequence that facilitates the efficient transcription of the inserted genetic sequence of the host. In one example, an expression vector contains an origin of replication, a promoter, as well as specific nucleic acid sequences that allow phenotypic selection of the transformed cells.

Recombinant: A recombinant nucleic acid is one that has a sequence that is not naturally occurring or has a sequence that is made by an artificial combination of two otherwise separated segments of sequence. This artificial combination can be accomplished, for example, by chemical synthesis or by the artificial manipulation of isolated segments of nucleic acids, e.g., by genetic engineering techniques. Similarly, a recombinant protein is one encoded for by a recombinant nucleic acid molecule.

Recombinase: A protein which catalyses recombination of recombining sites. Non-limiting examples of recombinases include CRE, FLP, Tn3 recombinase, transposon gamma/delta, and transposon mariner.

The cre and Flp proteins belong to the lambda/integrase family of DNA recombinases. The cre and Flp recombinases are similar in the types of reactions they carry out, the structure of their target sites, and their mechanism of recombination. For instance, the recombination event is independent of replication and exogenous energy sources such as ATP, and functions on both supercoiled and linear DNA templates.

Recombinases exert their effects by promoting recombination between two of their recombining sites. In the case of cre, the recombining site is a Lox site, and in the case of Flp the recombining site is a Frt site. Similar sites are found in transposon gamma/delta, TN3, and transposon mariner. These recombining sites include inverted palindromes separated by an asymmetric sequence. Recombination between target sites arranged in parallel (so-called “direct repeats”) on the same linear DNA molecule results in excision of the intervening DNA sequence as a circular molecule. Recombination between direct repeats on a circular DNA molecule excises the intervening DNA and generates two circular molecules. The cre/Lox and flp/frt recombination systems have been used for a wide array of purposes such as site-specific integration into plant, insect, bacterial, yeast and mammalian chromosomes has been reported (Sauer et al., Prvc. Natl. Acad. Sci. USA, 85:5166-70, 1988). Positive and negative strategies for selecting or screening recombinants are known.

Recombining site: A nucleic acid sequence that includes inverted palindromes separated by an asymmetric sequence (such as a transgene) at which a site-specific recombination reaction can occur. Examples include, but are not limited to, Lox, Frt (consists of two inverted 13-base-pair (bp) repeats and an 8-bp spacer that together comprise the minimal Frt site, plus an additional 13-bp repeat which may augment reactivity of the minimal substrate, TN3, mariner, and a gamma/delta transposon.

Selectable marker: A nucleic acid sequence that confers a selectable phenotype, such as in plant cells, that facilitates identification of cells containing the nucleic acid sequence. Transgenic plants expressing a selectable marker can be screened for transmission of the gene(s) of interest. Examples include, but are not limited to: genes that confer resistance to toxic chemicals (e.g. ampicillin, spectinomycin, streptomycin, kanamycin, geneticin, hygromycin, glyphosate or tetracycline resistance, as well as bar and pat genes which confer herbicide resistance), complement a nutritional deficiency (e.g., uracil, histidine, leucine), or impart a visually distinguishing characteristic (e.g., color changes or fluorescence, such as 13-gal).

Tapetum-specific gene: A gene sequence that is primarily expressed in the tapetum relative to the other tissues of the plant. Includes any tapetum cell-specific gene whose malfunction results in male-sterility. Examples include, but are not limited to: TA29 and TA13, pca55, pE1 and pT72, Bcp1 from Brassica and Arabidopsis (GenBank Accession Nos. X68209 and X68211), A9 from Brassicaceae (GenBank Accession No. A26204), and TAZ1, a tapetum-specific zinc finger gene from petunia (GenBank Accession No. AB063169).

Tapetum-specific promoter: A DNA sequence that directs a higher level of transcription of an associated gene in tapetal tissue relative to the other tissues of the plant. Tapetum is nutritive tissue required for pollen development. Examples include, but are not limited to the promoters associated with the genes listed under tapetum-specific genes.

Transduced and transformed: A virus or vector “transduces” or transfects” a cell when it transfers nucleic acid into the cell. A cell is “transformed” by a nucleic acid transduced into the cell when the DNA becomes stably replicated by the cell, either by incorporation of the nucleic acid into the cellular genome, or by episomal replication. As used herein, the term transformation encompasses all techniques by which a nucleic acid molecule can be introduced into such a cell. Examples include, but are not limited to, transfection with viral vectors, transformation with plasmid vectors, electroporation, lipofection, Agrobacterium-mediated transfer, direct DNA uptake, and microprojectile bombardment.

Transgene: An exogenous nucleic acid sequence. In one example, a transgene is a gene sequence, for example a sequence that encodes a cytotoxic polypeptide. In yet another example, the transgene is an antisense nucleotide, wherein expression of the antisense nucleotide inhibits expression of a target nucleic acid sequence. A transgene can contain native regulatory sequences operably linked to the transgene (e.g. the wild-type promoter, found operably linked to the gene in a wild-type cell). Alternatively, a heterologous promoter can be operably linked to the transgene.

Transgenic Cell: Transformed cells that contain a transgene, which may or may not be native to the cell.

Vector: A nucleic acid molecule as introduced into a cell, thereby producing a transformed cell. A vector can include nucleic acid sequences that permit it to replicate in the host cell, such as an origin of replication. Examples include, but are not limited to a plasmid, cosmid, bacteriophage, or virus that carries exogenous DNA into a cell. A vector can also include one or more cytotoxic genes, antisense molecules, and/or selectable marker genes and other genetic elements known in the art. A vector can transduce, transform or infect a cell, thereby causing the cell to express the nucleic acids and/or proteins encoded by the vector. A vector optionally includes materials to aid in achieving entry of the nucleic acid into the cell, such as a liposome, protein coating or the like.

Disclosed herein are methods for producing a perennial plant having an ablated floral apex resulting in floral or gametic deficiencies or an asexual phenotype and increased vegetative growth and growing season in switchgrass as well as other perennial plants produced by such methods, such as a floral-deficient or asexual perennial plant, and seeds produced by the parents of such plants. In one example, the method includes contacting a perennial plant with a vector, wherein the vector includes a construct to down regulate floral gene(s) operably linked to a plant promoter. Expression of this vector results in the production of asexual progeny, or a male or female floral deficient plant, or even total gametic (male or female or both) sterility, thereby producing a producing a perennial plant having increased vegetative growth. For example, the vector can be transfected into cells of the plant. Examples of plants that can be used include, but are not limited to, switchgrass, Atlantic coastal panic grass, big blue stem, poplar trees, sugar cane, and jatropha. In one example, the method results in an increase in vegetative growth and prolonged growing seasons.

The perennial plant having increased vegetative growth and biomass can have one or more desirable traits, such as two or more desirable traits, such as resistance to insects and other pests and disease-causing agents; tolerances to herbicides; post harvest activation of cellulase or other enzymes related to biofuel production methods; increased starch production; enhanced stability or yield; decreased lignin, increased cellulose; environmental tolerances; ease of hydrolysis, and ethanol production enhancements. The desirable traits can be linked to the gene which results in increased biomass through total floral sterility. In one example, the desired trait is due to the presence of a transgene(s) in the plant. In another or additional example, the desired trait is obtained through conventional breeding. In one example, the increased biomass trait is maintained through vegetative propagation. In another example the trait for increased biomass is produced as the outcome of a cross between two parents each with one component of the floral deficiency system. These parents can also carry one or more additional desirable traits.

Also disclosed are methods for producing a controlled total vegetative growth phenotype in perennial plants, as well as perennial plants produced by such methods, such as a male-deficient and/or female deficient perennial plant vegetatively propagated asexual plants, and seeds produced by parents of the plants that when crossed will produce an asexual or floral-deficient plant. In one example, the method includes crossing a first fertile plant having one or more desirable traits, such as two or more desirable traits, with second fertile plant. The second plant can also have one or more desirable traits. The first fertile plaint includes a vector, wherein the vector includes floral-specific promoter operably linked to a blocking sequence, such as a selectable marker, and recombining site sequences flank the blocking sequence. In addition, the vector includes a cytotoxic sequence, which is downstream to the promoter and the blocking sequence, and is in a position such that its expression is activated by the floral-specific promoter in the presence of a recombinase, which results in recombination at the recombining site sequences and removal of the blocking sequence. The second fertile plant includes another vector which includes a promoter operably linked to a recombinase. The promoter can be a constitutive promoter or an inducible promoter. If an inducible promoter is used, the second fertile plant is contacted with an inducing agent, before, during, or after crossing the first and second fertile plant. The inducing agent activates the inducible promoter, thereby permitting recombinase expression. If a constitutive promoter is used, the promoter will drive recombinase expression in the absence of an inducing agent. The expressed recombinase protein interacts with the recombining sites of the other vector, resulting in recombination, removal of the blocking sequence such that the floral-specific promoter is now operably linked to the cytotoxin, thereby driving expression of the cytotoxin in floral-specific tissues. The resulting progeny of such a cross are asexual or floral-deficient. In one example, the vector included in the second fertile plant also includes a promoter operably linked to a blocking sequence. The vector can be stably integrated into the genome of the plant.

In an alternative example, instead of using two vectors, all of the elements are placed on a single vector, which is transfected into plants or plant cells. The first and/or second plant can include one or more, such as two or more desirable traits. The first or the second fertile plant includes a vector. The vector includes a floral-specific promoter operably linked to a blocking sequence, such as a selectable marker, wherein the blocking sequence is flanked by a recombining site sequence, a cytotoxic sequence downstream of the blocking sequence such that the cytotoxic sequence is operably linked to the promoter upon recombination, and a promoter (such as a constitutive or inducible promoter) operably linked to a recombines. If an inducible promoter is used, the plant transfected with the vector is contacted with an inducing agent, before, during, or after crossing the plants. The inducing agent, or constitutively active promoter, promotes recombinase expression. The expressed recombinase protein interacts with the recombining sites, resulting in recombination, removal of the blocking sequence such that the floral-specific promoter is now operably linked to the cytotoxin, thereby driving expression of the cytotoxin. The resulting progeny of such a cross are asexual or floral-deficient. The vector can be stably integrated into the genome of the transfected plant.

Examples of site-specific recombination systems which can be used to practice the methods disclosed herein include the FLP/FRT system, the R/RS system and/or the CRE/LOX system. However, one skilled in the art will understand that other systems can also be employed.

Any floral-specific promoter can be used to practice the disclosed methods, including variants thereof that are functionally equivalent and confer gene express in or predominantly in floral tissues. Particular examples include, but are not limited to: floral-specific promoters, such as the FLORICA ULA/LEAFY homolog, anther-specific promoters, pollen-specific promoters, ovule-specific promoters, megasporocyte-specific promoters, megasporangium-specific promoters, integument-specific promoters, stigma-specific promoters, and style-specific promoters. In one example, floral-specific promoters include an embryo-specific promoter or a late embryo-specific promoter, such as the late embryo specific promoter of DNH1 or the HVA1 promoter; the GLB1 promoter from corn, and any of Zein promoter (Z27).

Cytotoxin molecules include traditional cytotoxins such as barnase, as well as antisense molecules, such as floral-specific gene antisense sequences, that decrease or inhibit the development of floral structures, thereby leading to sterility of the plant.

Examples of blocking sequences that can be used, include, but are not limited to, non-coding DNA sequences, and a selectable marker sequence. Any selectable marker can be used. Particular examples include, but are not limited to: genes that confer resistance to toxic chemicals such as the bar and pat genes which confer herbicide resistance, and those that impart a visually distinguishing characteristic, such as a color change. In addition, any cytotoxic sequence can be used to practice the methods disclosed herein, as long as the gene interferes with floral development, such as pollen or tapetal development, thereby rendering the plant sterile. Particular examples include, but are not limited to ribonucleases, such as barnase, as well as antisense sequences, such as a tapetum-specific antisense gene sequence.

Any constitutive or inducible promoter can be used. Examples of inducible promoters that can be used to practice the methods disclosed herein include, but are not limited to: heat shock promoters, glucocorticoid promoters, transcriptionally regulated promoters, chemically inducible promoters, and light activated promotes. Promoters regulated by heat shock, such as the promoter associated with the gene encoding the 70-kDa heat shock protein, increase expression several-fold after exposure to elevated temperatures. The heat shock promoter can be used as an environmentally inducible promoter for controlling transcription of a recombinase, such as FLP. Glucocorticoid promoters include a gene encoding glucocorticoid receptor protein (GR) which in the presence of a steroid hormone forms a complex with the hormone. This complex binds to a short nucleotide sequence (26 bp) named the glucocorticoid response element (GRE), and this binding activates the expression of linked genes. The light inducible system is exemplified by promoters that drive expression of various photosynthetic genes, such as some of the SSU, CAB, and PEP carboxylase promoters.

In contrast to inducible promoters, constitutive promoters fraction under most environmental conditions. Many different constitutive promoters can be utilized with respect to the methods of this disclosure. Exemplary constitutive promoters include, but are not limited to, promoters from plant viruses such as the 35S promoter from CaMV; promoters from such plant genes as rice actin; ubiquitin; pEMU; MAS and maize H3 historic; and the ALS promoter, a XbaI/NcoI fragment 5′ to the Brassica napus ALS3 structural gene or a nucleotide sequence with substantial sequence similarity. A particular example is a maize ubiquitin gene promoter.

### EXAMPLE 1

### Controlled Vegetative Growth in Switchgrass Results in Increased Vegetative Biomass

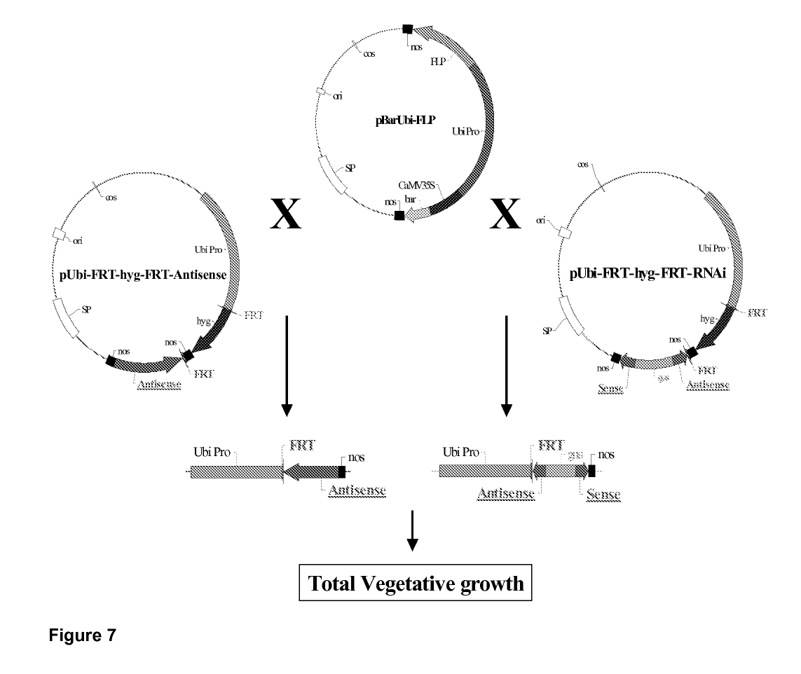

Total vegetative growth in switchgrass will result in increased biomass since no resources are directed toward flower development, in addition to providing a prolonged growing season. To achieve controlled vegetative growth in transgenic switchgrass, a two component system was used, whereby one genetic line (A) was crossed with a second transgenic line (B) to produce seed that will germinate but never flower. To achieve this total sterility in a progeny line (A×B) the lines (A) and (B) were constructed in various permutations of two examples as follows. In both methods, the first line (A) plants contain a construct expresses the FLP recombinase selected by expression of the bar gene for glufosinate resistance. In the first method line (B1) plants contained a construct in which the rice ubiquitin promoter and the antisense of the grass FLORICAULA/LEAFY homolog was separated by the hyg gene flanked by directly oriented FRT sites. This stable transgenic line (B1) may be selected by Hyg resistance driven by the ubiquitin promoter and will flower normally to produce seeds. The progeny of the cross between line (A) and (B1) resulting in the progeny (A×B1) the FLP recombinase should excise the blocking fragment (hyg gene) thus bringing together the ubiquitin promoter and the downstream antisense of the grass FLORICAULA/LEAFY homolog gene, turning off the FLORICA ULA/LEAFY homolog gene and giving rise to a total vegetative growth in the hybrid (FIG. 7).

In a second method using this same approach line (B2) plants contained a construct in which the rice ubiquitin promoter and a RNAi construct of the grass FLORICAULA/LEAFY homolog was separated by the hyg gene flanked by directly oriented FRT sites. This stable transgenic line (B2) can be also selected by Hyg resistance driven by the ubiquitin promoter and will flower normally to produce seeds. The progeny of the cross between line (A) and (B2) resulting in the progeny (A×B2) the FLP recombinase should excise the blocking fragment (hyg gene) thus bringing together the ubiquitin promoter and the downstream RNAi construct of the grass FLORICA ULA/LEAFY homolog gene, significantly down regulating the FLORICA ULA/LEAFY homolog gene and giving rise to a total vegetative growth in the hybrid (FIG. 7).

A transgenic bentgrass expressing recombinase FLP has been created. Agrobacterium-mediated plant transformation was used for delivery of gene constructs into switchgrass, creating transgenic lines containing the FRT-containing RNAi construction or the antisense of the turfgrass FLORICA ULA/LEAFY homolog gene into switchgrass. Using pSB 11-based binary vectors for chimeric gene construction, a reliable Agrobacterium-mediated grass transformation procedure that enables one to routinely produce transgenic switchgrass plants was established.

Cloning of the C-terminal region of the grass FLORICAULA/LEAFY homolog. Based on highly conserved sequences found in FLO/LFY and their homologs from other plant species, the following primers were synthesized to amplify the C-terminal region of the turfgrass FLO/LFY-like genes by the PCR. The 5′ primer was: 5′-TAC/TATA/CAAC/TAAA/GCCA/G/C/TAAA/GATG-3′ (SEQ ID NO: 5) and the 3′ primer was: 5′-AGCC/TTG/TGTG/TGGG/C/AACA/GTACCA-3 (SEQ ID NO: 6)′. Genomic DNA of bentgrass cv. Penn-A-4 was used as templates for the PCR. A PCR product of around 250 bp, as expected based on the FLO/LFY gene sequences, was isolated. This amplified DNA fragment, as part of the putative FLO/LFY-like genes candidate, was cloned into the EcoRV site of pLitmus28 (Biolabs) using standard protocols. This cloned fragment was sequenced and its homology to FLO/LFY homologs from other species was verified. This cloned fragment was also used as probe for Southern analysis to verify its identity and assess native gene structure in grasses. Northern analysis in different tissues may also be conducted to verify the expression levels of the FLORICA ULA/LEAFY homolog.

The isolated FLO/LFY homolog DNA fragment will be sufficient for use in the preparation of antisense and RNAi constructions. An alternative strategy would be to construct a cDNA library from the inflorescences of grass and screen for a FLO/LFY-homolog using the heterologous FLO/LFY-like gene from the monocot plants of rice or maize.

Production of transgenic switchgrass with a single-copy transgene insertion of the construction Ubi-FRT-hyg-FRT-Antisense and Ubi-FRT-hyg-FRT-RNAi. In order to obtain transgenic switchgrass plants whose total vegetative growth are controlled by FLP/FRT site-specific recombination, two gene constructs were prepared in which the rice ubiquitin promoter and the RNAi construction or the antisense of the grass FLO/LFY homolog was separated by the hyg gene flanked by directly-oriented FRT sites (FIG. 7).

To synthesize the antisense of the grass flower-specific gene-containing construct, pUbi-FRT-hyg-FRT-Antisense, the cloned C-terminal region of the grass FLO/LFY homolog will be released from the pLitmus28 by EcoRV digestion and ligated into the KpnI-SacI (bltmt-ended by Mung bean nuclease treatment) of the pSBUbi-FRT-hyg-FRT-gus. The orientation of the grass FLO/LFY homolog gene inserted by blunt-end ligation will be checked by sequencing, and the clone with the grass FLO/LFY homolog in reverse orientation (antisense), pUbi-FRT-hyg-FRT-Antisense, will be retained for further use (FIG. 7).

To synthesize the RNAi construct of the grass flower-specific gene, pUbi-FRT-hyg-FRT-RNAi, for expressing dsRNA in plant cells, we will first use pSBUbi-gus as a bridge vector, in which an 824 bp fragment of gus gene encoding [3-glucuronidase was placed in between the rice ubiquitin promoter and the nopaline synthase (nos) terminator. The cloned C-terminal region of the turfgrass FLO/LFY homolog may be released from the pLitmus28 by EcoRV digestion and placed upstream and downstream of the gus fragment in opposite directions, resulting in pSBUbi-gus-RNAi. Here, the gus fragment was used as a linker between gene-specific fragments in the antisense and sense orientations. The blocking DNA fragment, FRT-flanked hyg gene plus the nos terminator, FRT-hyg-FRT, may be released from pFRT-hyg-FRT as a SnaBI-KpnI fragment and ligated into the BamHI (flushed)-KpnI sites of the pSBUbi-gus-RNAi plasmid between the rice ubiquitin promoter and the downstream RNAi construction, giving rise to the final test vector pSBUbi-FRT-hyg-FRT-RNAi. Both Ubi-FRT-hyg-FRT-Antisense and Ubi-FRT-hyg-FRT-RNAi constructs will be then introduced into Agrobacterium tumefaciens LBA4404 by triparental mating or electroporation.

Simultaneously with the vector construction, mature seeds of switchgrass cv Alamo may be surface sterilized in 10% (v/v) Clorox® bleach plus two drops of Tween-2™ (Polysorbate 20) with vigorous shaking for 90 min. After rinsing five times in sterile distilled water, the seeds were placed onto callus-induction medium containing either 1) MS basal salts and vitamins, 30 g/l sucrose, 500 rag/l casein hydrolysate, 6.6 rag/l 3,6-dichloro-o-anisic acid (dicamba), 0.5 rag/l 6-benzylaminopurine (BAP) and 2 g/l Phytagel; or, 2) MS basal salts and vitamins, 30 g/l maltose, 22.5 uM 2,4 dichlorophenoxyacetic acid (2,4-D) 0.5 uM 6-benzylaminopurine ˜BAP) and 2 g/l Phytagel. The pH of the medium may be adjusted to 5.7 before autoclaving at 120° C. for 20 min. The culture plates containing prepared seed explants were kept in the dark at room temperature (27 C) for 4-6 weeks. Embryogenic calli is visually selected and subcultured on fresh callus-induction medium in the dark at room temperature at 27° C. every 2-4 weeks and for 1 week before co-cultivation.

The above two constructs were separately introduced into switchgrass (cv Alamo) by Agrobacterium-medicated transformation using embryogenic callus. For plant regeneration, transformed and selected callus is transferred to MS medium containing 1.4 μM gibberillic acid (GA3), incubated at 27° C. with a 16-h photoperiod. The regenerated plants were then transferred into soil and grown in the greenhouse. Molecular characterization of these T₀ transformants were conducted to demonstrate the presence and expression of the introduced foreign genes, and determine the copy number of transgene insertion. Southern blot analysis may be performed on the turf transformants. Genomic DNA may be obtained from leaves using procedure described in QIA amp Tissue Kit (available from QIAGEN, Inc., Chatsworth, Calif.) for Southern analysis using either, hyg gene as probes following standard molecular biology techniques. Transgenic plants with single-copy transgene insertion will be selected for further analysis. Also, we will isolate total RNA from leaf tissues of positively identified transgenic plants and determine mRNA accumulation in separate transformants. The RNeasy Plant Total RNA Kit were used to simplify RNA isolation procedures (QIAGEN Inc., Chatsworth, Calif.). Ten μg total RNA may be fractionated on agarose gels in denaturating conditions (7.5% formaldehyde) for Northern blot analysis.

Using standard molecular biology techniques, it is anticipated that one will be able to make the two designed gene constructs and introduced into switchgrass through Agrobacterium-mediated transformation, producing transgenic switchgrass plants. No problems were anticipated for doing this, since reliable transformation systems have been established in other grasses that have allowed us to routinely transform bentgrass with high efficiency.

Cross-pollination with recombinase FLP-expressing plants to produce a hybrid with total vegetative growth. In order to check whether FLP-mediated excisional DNA recombination will excise the blocking fragment (hyg gene) thus bringing together the ubiquitin promoter and the downstream RNAi construction or the antisense of the grass FLORICAULA/LEAFY homolog gene, turning off the FLORICA ULA/LEAFY homolog gene and giving rise to a total vegetative growth, the flowered transgenic plants containing the antisense or RNAi constructs will cross-pollinated with pollen the FLP-expressing homozygous transgenic plants that has been obtained before. Since the antisense or RNAi-containing _{T0} plants are hemizygous with respect to transgene inserted, there is only 50% of the hybrid that contain transgenes. Those hybrid plants may be identified using PCR to verify the presence of the rice ubiquitin promoter. These plants may then be grown in the greenhouse and vernalized. Their total vegetative growth may be examined in comparison with wild-type plants. Southern analysis may be conducted to check the occurrence of FLP-mediated excisional DNA recombination, and Northern analysis may be performed with RNA from inflorescences to check the down-regulation of the FLORICAU˜LA/LEAFY homolog gene. Based on the results obtained, the transgenic lines that have shown total vegetative growth for producing homozygous plants were chosen.

These plants have been used to demonstrate the effectiveness of the total vegetative growth for increasing biomass genetically modified switchgrasses and other perennial plants used for biofuels.

### EXAMPLE 2

### Expression of Antisense its or Barnase in Switchgrass Results in Male Sterility

This example describes methods used to develop transgenic male sterile switchgrass. Similar methods can be used to produce other transgenic male sterile perennials. The male sterile plants produced prevent outcrossing thus prolonging vegetative growth in plants such as switchgrass that are obligate outcrossers. Briefly, switchgrass cells are transformed with DNA sequences that cause herbicide resistance and male sterility.

### Generation of Plasmids

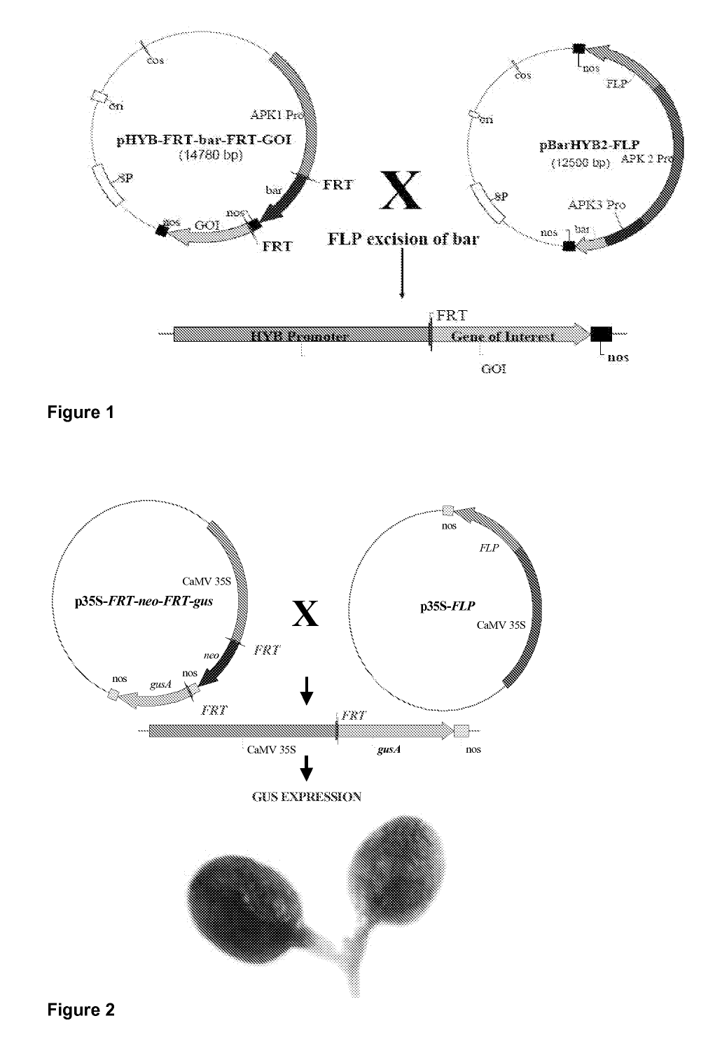

To induce male-sterility in creeping bentgrass (Panicum virgatum L. cv Alamo), constructs containing an antisense rice tapetum-specific gene (rts) gene (FIG. 1A), or a ribonuclease gene from Bacillus amyloliquefaciens called barnase (FIG. 1B), were introduced separately into switchgrass embryogenic cultures using Agrobacterium tumefaciens-mediated transformation as described below. A set of pSB 11-based Agrobacterium binary vectors for turfgrass transformation with the antisense of a rts (sense sequence shown in SEQ ID NO: 1) and a ribonuclease gene from Bacillus amyloliquefaciens, called barnase, both under the control of a rice tapetum-specific promoter TAP (original driving rts gene, SEQ ID NO: 2). To synthesize the antisense rts- and barnase-expression vectors containing the herbicide-resistant bar gene as a selectable marker, the HindIII-BamHI fragment (corn ubiquitin promoter) from pAHC27 were cloned into respective sites of the pSBbarB to replace the original 35S promoter, generating pSB-UbibarB. The antisense rts gene and barnase gene expression cassettes were then be added to obtain the final male sterility-inducing vectors shown in FIGS. 1A and B. To generate the antisense rts expression vector, the PvuII fragment from pTAP (anti-sense) including the tapetum-specific promoter TAP driving antisense rts gene was ligated into the flushed HindIII site of pSB-UbibarB. To generate the barnase gene expression vector, a 0.52 kb barnase coding sequence from pMT416 was amplified by PCR using primers:

### 5′ CACAGGAAACAGGATCCGCGG-3′ (SEQ ID NO: 3) and

5′ CGCGAGCTCGCCGGAAAGTGAAATTGACC-3′ (SEQ ID NO: 4) (underlined is the SstI restriction site). The amplified PCR product was digested with SstI and cloned into pTAP/gus after gus coding fragment being removed by SmaI/SacI digestion, generating pTAP/barnase. The tapetum-specific promoter TAP driving barnase gene was released from pTAP/barnase as a PvuII fragment and ligated into the flushed HindIII site of pSB-UbibarB. The vectors are designated as p115 for the antisense rts gene (FIG. 1A) and p127 for the barnase gene (FIG. 1B).

Because the barnase gene- and the antisense rts gene were under the control of the rice tapetum-specific promoter TAP, the antisense rts gene and barnase gene were expressed in tapetal cells and microspores of the transgenic switchgrass.

### Transformation of Plants

Several systems can be used to transform switchgrass plant cells. The methods disclosed herein are not limited to any particular transformation method. Methods that can be used to transform various grass species (such as switchgrass, creeping bentgrass, tall fescue, perennial rye grass, Bermuda grass, and Kentucky blue grass) include, but are not limited to, biolistics, Agrobacterium, and whisker-mediated transformation. A strain similar to the Agrobacterium superbinary system was used with a tissue culture approach for selection of bar gene expression in transformed Agrostis pahlstris (cvs Penn A4) and switchgrass (Panicum virgatum L. cv Alamo), cells. The plasmids with gene constructs of interest were introduced into Agrobacterium tumefaciens strains LBA4404 (containing co-integrative vector pSB 111) by triparental mating or electroporation. The two plasmids co-integrate by homologous recombination in Agrobacterium tumefaciens cells.

Mature seeds of creeping bentgrass (cultivars Penn A4) switchgrass (Panicum virgatum L. cv Alamo), were surface-sterilized and plated on callus induction media (modified MMSG or MSA2D media). The plates were kept in the dark at room temperature (RT or 27 C) for 3-6 weeks. The proliferating calli were selected and transferred to new maintenance medium on a regular basis. Only callus that is friable, embryogenic and regenerable is used for transformation. The chosen callus was transferred to fresh medium prior to co-cultivation with Agrobacterium to promote active cell division. This callus was used for transformation within a week after transferring to new plates.

Agrobacterium tumefaciens with was induced with acetosyringone as follows:

Agrobacterium tumefaciens LBA4404, harboring male sterility vectors were streaked from a glycerol stock and grown at 28° C. on plates containing AB medium, supplemented with 10 μg/ml tetracycline and 50 μg/ml spectinomycin. After three to six days, the cells were scraped from the plate and suspended in Agrobacterium growth medium containing 100 μM acetosyringone, and grown to an OD₆₆₀ of about 0.1-0.5. The bacterial suspension was incubated at 25° C. in the dark with shaking for 3.5 hours before using it for co-cultivation.

Friable callus (0.001 mg-100 g) was mixed with the pre-induced Agrobacterium suspension and incubated at room temperature in the dark for 1.5 hours. The contents were poured into a sterile Buchner-funnel containing a sterile Whatman filter paper. Mild vacuum was applied to drain the excess Agrobacterium suspension. The filter was moved to a plate containing maintenance medium supplemented with 100 μM acetosyringone, and the plate stored in the dark at room temperature for three days. Subsequent to the three day co-cultivation, the co-cultivated calli were rinsed with 250 μg/ml cefotaxime to suppress bacterial growth, and the calli placed on agar plates containing maintenance medium which included 15 mg/L PPT (phosphinothricine, for bar selection) and 250 μg/ml cefotaxime. The calli were kept in the dark at RT for 6-8 weeks and checked periodically for proliferation of the calli on the 15 mg/L PPT. Subsequently, the PPT-resistant calli were placed on regeneration medium containing PPT and cefotaxime. The proliferating calli were first moved to Regeneration Medium I containing cefotaxime (Research Products International Corp.) and PPT (Duchefa Biochemie, B.V.). The tiny plants were separated and transferred to deep peWi plates containing Regeneration Medium II to promote root growth. PPT and cefotaxime were included in the medium to respectively maintain selection pressure and kill any remaining Agrobacterium cells. After 2-3 weeks, or when the plants were 1.5-2 cm tall, they were moved to plant-cons containing MSO II without antibiotics. When the plants were about 10 cm tall and develop extensive root systems, they were transferred to soil and grown for 3-4 weeks with 12 hours light/day. The plants were then transferred to 6-inch pots in the greenhouse, where the temperature is maintained between 21-25° C. Supplemental lighting can be added to increase timing of light exposure for flowering.

### Generation TO Male Sterile Transformants

The transgenic plants were vegetatively propagated and increased. The TO plants produced seeds by backcrossing to the recipient variety and outcrossing to other cultivars for transmission of the transgenic traits.

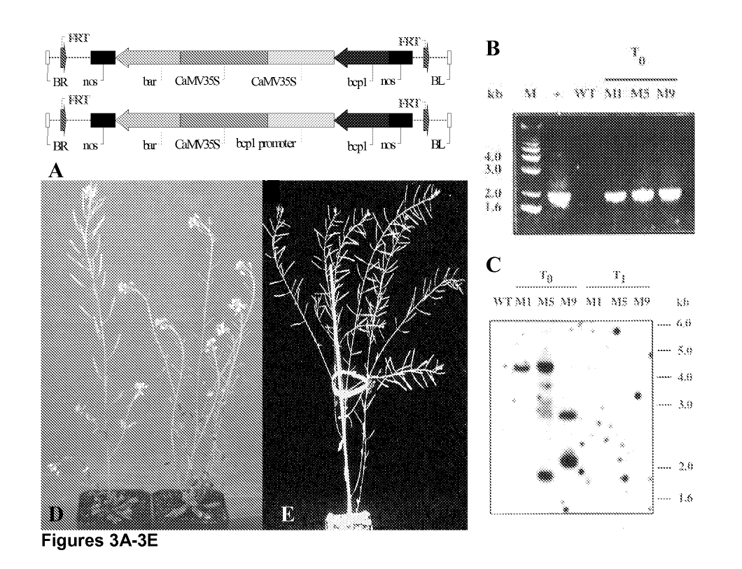

Transformants were screened for glufosinate resistance by ‘paint assays’ to leaves and subsequently analyzed by standard molecular procedures (PCR and Southern blotting) to characterize the insertion events in the regenerating TO plants and their stability in subsequent generations. The plants were sprayed with 0-100% v/v of liberty or finale (Aventis Corp.) and shown to be resistant to the herbicide (FIG. 2). Southern analysis using the bar gene and/or barnase as probes revealed transgene insertion in the male-sterile plants. Therefore, stable transformation of creeping bentgrass was achieved, as evidenced by the resistance of the plants to herbicide (due to the presence of the bat gene) and the male sterility due to the presence of the male sterile constructs.

### Developmental and Phenotypic Analysis of Pollen Development and Viability

The herbicide-resistant male sterile TO plants had normal vegetative growth and morphology in comparison to non-transgenic tissue culture regenerated plants. As described above, transformation of herbicide tolerant creeping bentgrass and switchgrass (Panicum virgatum L. cv Alamo) was achieved. All transgenic plants were linked to one or the other male sterility construct (p115 or p127, FIGS. 1A and 1B) as shown by macrophotography and light microscopy. In addition, flowering herbicide resistant male sterile TO plants had normal vegetative growth and morphology in comparison to non-transgenic tissue culture regenerated plants (FIG. 3A), except the anthers were shrunken (FIG. 3B) and the pollen was aborted prior to the starch filling stage as indicted by IKI2 (iodine) staining (FIG. 3C). Pollen was obtained from transfected and control plants, and the viability determined by staining with iodine (IKI2) and examination by microscopy, using methods known to those skilled in the art. Wild-type pollen was heavily stained with IKI2, indicating that the pollen was filled with starch and viable. Pollen viability for wild-type plants was between 30-85%. In contrast, transgenic plants (plants transfected with P127 or p 115, FIGS. 1A and 1B) had no visible IKI2 staining, indicating that the pollen was not filled with starch, and thus not viable (FIG. 3C). In addition, fewer pollen grains were observed in the male sterile plants. Out of 174 p115 transformed plants, 79 plants flowered, and 40 of them were sterile. Out of 47 p127 transformed plants, 11 flowered and 10 of these were sterile. Pollen fertility was determined using several methods, including in vitro pollen germination analysis, in vivo pollen tube studies, and a fertility test to nontransgenic varieties analyzed for glufosinate resistance.

These plants have been used to demonstrate the effectiveness of using male sterility and thus increasing and prolonging vegetative growth for increasing biomass genetically modified switchgrasses and other perennial plants used for biofuels.

### EXAMPLE 3

### Generation of T1 Plants Yields Male Sterile Plants which Segregate with the Bar Gene can be Crossed with Female Sterile Plants to Produce Totally Sterile Progeny Plants that Show Increased Biomass and Prolonged Vegetative Growth

First, to generate T1 plants, the sterile male TO plants generated above were crossed with fertile wild-type switchgrass. The bar gene segregated in Mendellian ratios of approximately 1:1. One-half of the T1 plants were also male sterile and one-half were male fertile as evidenced by the absence of starch accumulation in the pollen in about 50% of the T1 plants. Wild-type T1 plants exhibited 70-95% pollen viability, while male sterile T1 plants exhibited >0.001% (p115) or 0.1-0.01% (p127) viability. Flower development was observed in male sterile plants in comparison to wild type plants by light microscopy. The flowers and the anthers in T1 male sterile plants appear normal with respect to the wild type except that they do not undergo the starch filling phase of pollen maturation. The PCR results on the T1 male sterile plants demonstrated the presence of the introduced constructs encoding the bar gene and the male sterility genes. The male sterile plant phenotype segregated with the bat” gene as evidenced by herbicide resistance and the expression of the male sterility genes. Since the bar gene segregated with the tapetum-specific promoter driving male sterile expression as a dominant Mendellian characteristic, overseeding can be easily used by consumers with proper management.

### Breeding Through Backcrossing to Wild-Type Female and Analysis on Hemizygous T2 Transformants

The resulting progeny from backcrossing T1 plants will be hemizygous for the bar selectable marker as well as the male sterile phenotype. Molecular analysis (such as PCR and Southern hybridization) can be conducted to confirm ability for transmission of the male sterile trait. These plants have been used to demonstrate the effectiveness of using male sterility and thus increasing and prolonging vegetative growth for increasing-biomass genetically modified creeping bentgrass, wheatgrass, switchgrass, and other perennial plants used for biofuels.

A second line can be developed that is female sterile such that by crossing these two lines the resulting progeny will be totally sterile and result in increased biomass production and prolonged growing seasons.

### EXAMPLE 4

### Site-Specific Recombination Used to Generate Lines Used for Increasing Biomass in Perennials

Disclosed is a method for using site-specific recombination to generate plants with controlled total vegetative growth in offspring from fertile, transgenic parents. The resulting second-generation offspring exhibited total vegetative growth and produced no viable seed. In addition, this technology allows the capability of deleting unwanted DNA sequences (such as antibiotic resistance markers) from transgenic plants. This example describes methods using the FLP/FRT recombinase system. However, one skilled in the art will understand that alternative recombinase systems can be used, such as the CRE/LOX or RS systems.

Site-specific recombinases are enzymes that recognize specific DNA sequences, and in the presence of two such recombination sites they catalyze the recombination of DNA strands. In the FLP/FRT system, for example, FLP can catalyze excision/integration or inversion of DNA fragment according to the orientation of the two FRT sites. Recombination between directly oriented sites leads to excision of the DNA between them, whereas recombination between inverted target sites causes inversion of the DNA between them. Some site-specific recombination systems do not require additional factors for their function and are capable of functioning accurately and efficiently in various heterologous organisms. For example, FLP/FRT from the 2 gm plasmid of Saccharomyces and Cre/lox from E. coli phage P1 efficiently catalyze DNA recombination in dicot and monocot plant cells.

### Plant Expression Cassettes for Site-Specific Recombination

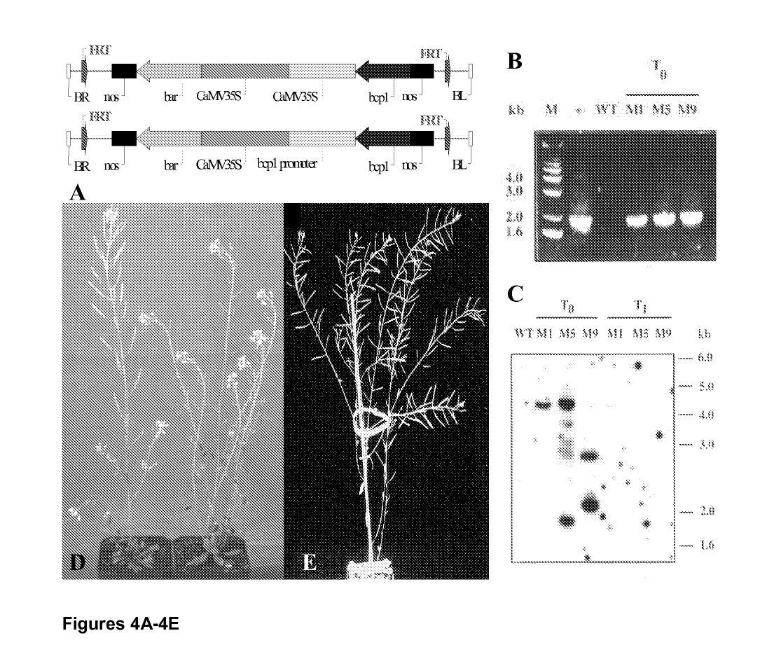

Two pSB 11-based Agrobacterium binary vectors for plant transformation can be generated as follows (FIG. 4). The first, the vector containing FRT sites, includes a floral-specific promoter, the FRT and FRTm sequences which flank a blocking DNA fragment (such as a selectable marker), followed by a cytotoxic gene (such as a barnase sequences or an antisense sequence). The cytotoxic gene was therefore separated from the promoter by the blocking DNA fragment bracketed by recombination targeting sequences The second vector, the FLP recombinase vector, includes an FLP recombinase driven by a promoter, such as a constitutively active promoter (for example the rice ubiquitin promoter, rice Actin 1, corn ubiquitin promoter, and the 35S CaMV promoter) or an inducible promoter. Examples of selectable markers included three bar or pat genes which can be driven by an Ubiquitin corn promoter (Ubi-c) with a nos 3″ termination signal. Examples of inducible promoters include, but are not limited to, those responsive to environmental stimuli. Ideally, an inducible promoter is highly inducible by agents or signals that can readily penetrate the seed or other plants parts, is non-toxic, inexpensive, environmentally acceptable, and its expression essentially ‘non-leaky’. One skilled in the art will understand that other recombinase systems can be used. For example, Cre can be substituted for FLP and Lox can be substituted for FRT.

To synthesize this portion of the FRT vector containing the herbicide-resistant bar gene as a selectable marker, the HindIII-BamHI fragment (corn ubiquitin promoter) from pAHC27 is cloned into respective sites of the pSBbarB to replace the original 35S promoter, giving rise to pSB-UbibarB. The 34 bp FRT sequence was blunt end ligated to the 5′ end of the pSB-UbibarB and the FRTm sequence added to the 3′ end. To synthesize the FLP-expression vector, the modified FLP gene is released as a SalI (flushed with DNA Polymerase I, ICdenow fragment, Biolabs)-SacI fragment from the plasmid JFLO and ligated into the BamHI (flushed with DNA Polymerase I, Klenow fragment, Biolabs)-SacI sites of the plasmid pEH30 to replace the original gusA gene. The modified FLP gene included a plant consensus sequence around the ATG codon. The HindIII-EcoRI fragment containing the FLP gene driven by the inducible promoter was released, flushed with DNA Polymerase I, Klenow fragment and ligated into the vector described above giving rise to the final construct.

FLP expression in the presence of the inducing agent caused excision of the FRT flanked sequences juxtaposing the promoter in the FRT vector and the cytotoxic gene rendered the resultant plant sterile.

### Methods for Increasing Biomass and Prolonging Vegetative Growth

Each plasmid was transfected into a different plant (for example using Agrobacterium tumefaciens LBA4404 produced by triparental mating or electroporation as described in Example 1 to infect plant cells which are then selected by resistance to the selectable marker bar), and the resulting TO fertile herbicide-resistant transgenic plants were selfed and/or crossed with fertile wild type plants. The plants containing the FLP vector are also either selfed and/or crossed with fertile wild type plants. The resulting T1 plants segregated according to Mendiallian ratios. The homozygous T1 plants were herbicide resistant and were crossed as parents. The progeny can be exposed to the inducer prior to, during or after crossing, to drive expression of the recombinase. The resulting progeny will receive both promoters, such that when recombinase expression was induced in the progeny, the recombinase removes the FRT-flanked blocking DNA sequence (such as a selectable gene, or noncoding DNA), bringing together the promoter and the cytotoxic sequence. The expression of the cytotoxic sequences produces transgenic seeds that germinated, produced a vegetative cycle and never produced fertile flowers.

### Linked Regulation

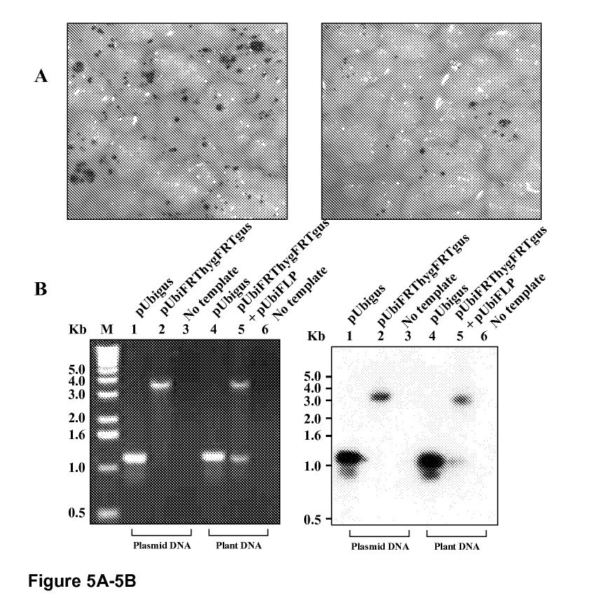

In order to avoid variation that may be introduced by crossing the FLP function in during the last pollination during seed production, the entire recombinase system can be designed to be in one expression cassette as shown in FIGS. 5-8. In this method, the recombinase (such as FLP recombinase) was under promoter (such as a chemical inducer as shown in FIG. 5) and operably linked to a cytotoxic gene, such as barnase. In one particular example, the promoter was a floral apex specific promoter, such that its expression would result in ablation of the entire inflorescence resulting in total infertility.

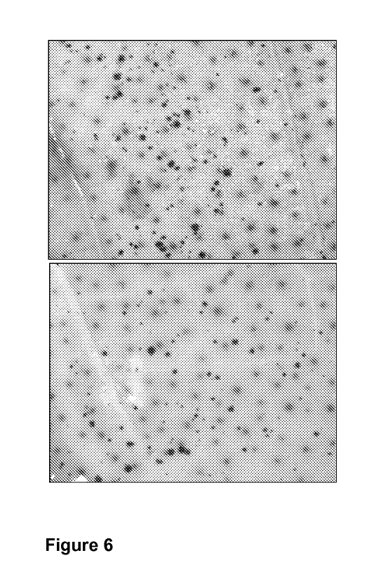

In addition, methods for elimination of invasive plants, by creating a bio-herbicide were disclosed which permit the ability to safely eliminate an invasive plant against a stable ecological background without effects on other plants. The promoter shown in FIG. 6 is an embryo specific promoter (Glb1) from maize, such that after induction the blocking sequence is removed, and permits expression of the cytotoxic gene through the Glb1 promoter. The seeds produced by such a transgenic plant are sterile. Induced transgenic seeds can be sown as a bioherbicide to control invasive plants and weeds without the use of chemicals.

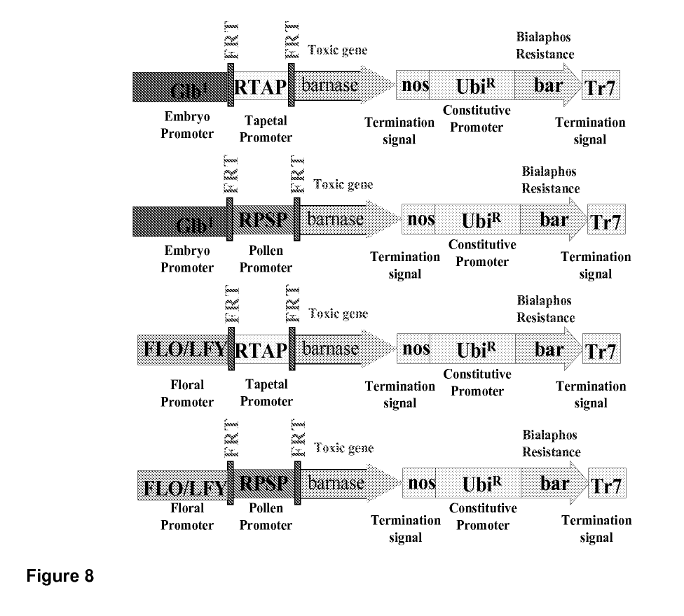

Another variation shown in FIGS. 7 and 8 depicts a light inducible promoter, such that the recombinase is activated after the seed germinates, excising the blocking sequence, such that the pollen carries the cytotoxic sequence. In another example, the transmitted poll can kill the entire plant, in this case the cytotoxic gene could be any element which is systemic and lethal. The systemic portion can be directed by a movement protein (such as the TMV movement protein). The cytotoxic gene can be activated by an inducible promoter, such as a chemically inducible promoter. If this is an environmentally acceptable inducible promoter, only the pollinated plants of the specific species are eliminated when the promoter is activated.

### EXAMPLE 5

### Controlled Vegetative Growth in Switchgrass Results in Increased Vegetative Biomass by Floral Ablation

Total vegetative growth in switchgrass will result in increased biomass since no resources are directed toward flower development, in addition to providing a prolonged growing season. To achieve controlled vegetative growth in transgenic switchgrass, a two-component system was used whereby one genetic line (A) was crossed with a second transgenic line (B) to produce seed that will germinate but never flower. Those versed in the art would also be able to configure the system such that it was contained in a single component and driven by a promoter that can be induced by the application of an exogenous signal.

In the two-component system, to achieve asexuality in a progeny line (A×B) the lines (A) and (B) can be constructed in various permutations of the example as follows. The first line (A) plants contains a construct that constitutively, or by induction, expresses the FLP recombinase selected for by co-expression of the bar gene for glufosinate resistance. Parental line (B) plants contain a construct in which the switchgrass FLORICA ULA/LEAFY homolog promoter is separated from a cytotoxic sequence (e.g., barnase) by the hyg gene flanked by directly oriented FRT sites. This stable transgenic line (B) can be selected by Hyg resistance driven by the ubiquitin promoter and will flower normally to produce seeds. The progeny of the cross between line (A) and (B) resulting in the progeny (A×B) the FLP recombinase should excise the blocking fragment (hyg gene) thus bringing together the FLORICAULA/LEAFY homolog promoter and the downstream cytotoxic sequence (barnase). In the A×B progeny the cytotoxic gene will express specifically in early floral meristem development and will result in the ablation of the cells within which the developmental transition occurs. Such a system will prevent the establishment of floral tissues and result in a perennial plant that is constitutively vegetative and incapable of sexual reproduction. Such a plant would have a phenotype of increased biomass production and total gene containment.

In light of the foregoing, it will now be appreciated by those skilled in the art that various changes may be made to the embodiment herein chosen for purposes of disclosure without departing from the inventive concept defined by the appended claims. Non limiting examples of such changes including using

We claim:

1. A method of producing a perennial plant comprising contacting the plant with a vector or vectors, wherein an outcome comprises a perennial plant having increased biomass.

2. The method of claim 1, wherein the outcome comprises a perennial plant having controlled vegetative growth.

3. The method of claim 1, wherein the perennial plant having controlled vegetative growth has increased biomass and prolonged growing seasons.

4. The method of claim 1, the perennial plant is a switchgrass (Panicum virgatum L.).

5. The method of claim 1, wherein the method produces a viable seed that will germinate without producing fertile flowers.

6. The method of claim 1, wherein the method produces floral deficiencies by down regulation of a floral specific gene(s).

7. A method of producing controlled vegetative growth in a perennial plant comprising:
crossing a first fertile plant having a desirable trait(s) with second fertile plant, wherein the first fertile plant comprises a first vector comprising a constitutive promoter operably linked to a blocking sequence, wherein the blocking sequence is flanked by a recombining site sequence, and a sequence involved with floral fertility, wherein the second fertile plaint comprises a second vector comprising a promoter operably linked to a recombinase; and
permitting expression of the recombinase, wherein crossing the first and second fertile plant results in production of a viable seed that will germinate to produce an asexual or floral deficient perennial plant resulting in total vegetative growth, increased biomass, and a prolonged growing season.

8. A method of producing controlled vegetative growth in a perennial plant comprising:
crossing a first fertile plant having a desirable trait(s) with a second fertile plant, wherein the first or the second fertile plant comprises a vector, wherein the vector comprises a constitutive-promoter operably linked to a blocking sequence, wherein the blocking sequence is flanked by a recombining site sequence, a floral specific sequence downstream of the blocking sequence such that the floral specific sequence causes down regulation and is operably linked to the promoter upon recombination, and a promoter operably linked to a recombinase; and
permitting expression of the recombinase, wherein crossing the first and second fertile plant results in production of a viable seed that will germinate to produce a plant with total vegetative growth in a perennial plant.

9. The method of claims 7 or 8, wherein the promoter operably linked to the recombinase is a constitutive promoter.

10. The method of claims 7 or 8, wherein the promoter operably linked to the recombinase is an inducible promoter, and wherein the method further includes contacting the second fertile plant with an inducing agent to permit expression of the recombinase.

11. The method of claim 10, wherein the second fertile plant is contacted with the inducing agent before crossing with the first fertile plant.

12. The method of claim 10, wherein the promoter operably linked to the recombinase is an inducible promoter, and wherein the method further includes contacting the first or second fertile plant with an inducing agent to permit expression of the recombinase.

13. The method of claim 10, wherein the seed from the crossing of the first and second fertile plants is contacted with the inducing agent.

14. The method of claims 7 or 8, wherein the second vector further comprises a promoter operably linked to a selectable marker.

15. The method of claims 7 or 8, wherein the recombinase is an FLP recombinase and the recombining site sequence is an FRT sequence.

16. The method of claims 7 or 8, wherein the recombinase is an CRE recombinase and the recombining site sequence is an LOX sequence.

17. The method of claims 7 or 8, wherein one vector comprises a floral-specific construct produced from the FLORICAULA/LEAFY homolog.

18. The method of claim 7 or 8, wherein the floral-specific vector comprising a FLORICAULA/LEAFY homolog is an antisense construct.

19. The method of claims 7 or 8, wherein the floral-specific construct comprises a FLORICAULA/LEAFY homolog is an operable RNAi construct.

20. The method of claim 2, wherein an amount of viable flowers produced is less than 0.01% as compared to a wild-type perennial plant of a same variety as the perennial plant having increased biomass.

21. The method of claim 2, wherein an amount of viable flowers produced is less than 0.001% as compared to a wild-type perennial plant of a same variety as the perennial plant having increased biomass.

22. The method of claim 2, wherein the perennial plant having increased biomass comprises one or 10 more desirable traits.

23. The method of claim 22, wherein the desirable traits are selected from the group consisting of herbicide resistance, decreased lignin, increased cellulose, post-harvest induced cellulase synthesis, drought tolerance, pest and disease resistance.

24. The method of claim 22, wherein the one or more desirable traits is linked to increased biomass.

25. The method of claim 1, wherein increased biomass is maintained through vegetative propagation.

26. A perennial plant produced by the method of claim 1.

27. A totally-sterile perennial plant produced by the method of claim 2.

28. A seed of the plants of claims 7 or 8.

29. The seed of progeny of plants of claims 7 or 8.

30. A seed of the progeny of crosses between the first and second plants of claim 7 or 8.

31. The method of claims 7 or 8, wherein the blocking sequence is a selectable marker gene sequence.

32. The method of claim 3, wherein the selectable marker is a hyg, gat, bar or pat gene sequence.

33. The method of claim 10, wherein the inducible promoter is a heat shock promoter, a chemically inducible promoter, or a light activated promoter.

34. The method of claims 7 or 8, wherein the recombinase is integrated in the genome of the second fertile plant.

35. The method according to claims 7 or 8, wherein the second fertile plant has one or more desirable traits.

36. The method of claim 7 or 8, wherein the first plant comprises one or more transgenes that confer the desirable trait(s).

37. The method of claim 1, wherein increased biomass is maintained through vegetative propagation.

38. The method of claim 1, wherein the outcome comprises a perennial plant having herbicide resistance.

39. The method of claim 1, wherein the outcome comprises a perennial plant having rough tolerance.

40. The method of claim 1, wherein the outcome comprises a perennial plant having salt tolerance.

41. The method of claim 1, wherein the outcome comprises a perennial plant having cold tolerance.

42. The method of claim 1, wherein the outcome comprises a perennial plant having increased photosynthetic efficiencies.

43. The method of claim 1, wherein the outcome comprises a perennial plant having enhanced cellulose production.

44. The method of claim 1, wherein the outcome comprises a perennial plant having increased root growth.

45. The method of claim 1, wherein the outcome comprises a perennial plant having enhanced phosphate uptake.

46. The method of claim 1, wherein the outcome comprises a perennial plant having increased leaf angle.

47. The method of claim 1, wherein the outcome comprises a perennial plant having decreased lignin content.

48. The method of claim 1, wherein the outcome comprises a perennial plant having increased shade tolerance.

49. The method of claim 1, wherein the outcome comprises a perennial plant having broad fungal resistance.

50. The method of claim 1, wherein the outcome comprises a perennial plant having broad insect resistance.

51. The method of claim 1, wherein the outcome comprises a perennial plant having stay green characteristics.

52. The method of claim 1, wherein the outcome comprises a perennial plant having delayed senescence.

53. The method of claim 1, wherein the outcome comprises a perennial plant having decreased nitrogen utilization.

54. The method of claim 1, wherein the outcome comprises a perennial plant having increased free sugars, especially sucrose.

55. The method of claim 1, wherein the outcome comprises a perennial plant having enhanced endophyte growth.

56. The method of claim 1, wherein the outcome comprises a perennial plant having prolonged growing season.


SEQUENCE LISTING (6 sequences)

SEQ ID NO: 1 - DNA, 1753 bp, Oryza sativa
gagctcaccggcgaggcggtgcgtctcctcggagatgtggtagaagctggcgccccatttcatggcggcgagcgggccgggcggcctcctgccgcagcgggattcggtggctagaccgatctgtgggtggaggacggggacgaggtagaggagacagaggcggcattggaagaggggaagaggaggaggaagtggtggcaggcagaggcggatgaggaacttgcgccagcgacgtggatatggagggggcgacggcaatggggaggcggcgatggaagcgaggagatgggcaggcggcggaggcagcggtggattttttttttctttttctttttcggaccctttaccctgctcggtgattcttcttttttatacagcacgacggcttctcctattcacgacgcctcggctggaccatggaccgttggccactggagcattcttccatgatctagattttttttttcactcaactttactacttcacatctgatggctggtgttgaattcattgtgcatccaacggtcattattaaattgatgacgtggcgcaatgaggtgacgaaacactttactttttttactactttagatctgtcggcaggagtcccagatagatacttgagctggttagttgggttttggatggagtaactttctgcagactgcaacattctgacacacgtagcagcacaaaagagttgcgaacaaacttggactgttaacatgtcaacgcataaaactgaaaaaaaaaacctgtcaaaatgcataataaataaaactgaaaaaaaataagaataaatgttgagagtgggatttgaacccacgccctttcggaccagaaccttaatctggcgccttagaccaactcggccatctcaactttttgctctgtcatccaaacaaagttataagaaatcatataataataactaagacttgatgcctcagtagtttagttaaactaatttgaatttgttagtacagtttgcatttcaaattgttccaatttggacgccacggctggtttcagttgctcacgacgcctcacacacatattttgcttccttgcttgtgacactagggcacaaaactccaacactcaaacgacacttcacgcatctctcctgaaatcttgcaccccccaactctgcatcgtcgcgtataaaatgcagaccaaaccccagctcaactctgcatcatcatcatcaactcgatagaaaaagaaagaaattaaaaagaaaatcacggcgcgtgagcttgcagagacagcaatggtgagagttgctgccgccgcggcggtgctcgtgctggcggcggcggcggcggcggcggcggccatggccgccgagccgcccaccgatgacggcgcggtccgggtggcggcggggctgacgaagtgcgtgtccgggtgcggtagcaaggtgacctcctgcttgctcggctgctacggcggcggcggcggcgccgccgccgccgcgacggcgatgccgttctgcgtcatcggctgcaccagcgacgtcttgtcctgcgccaccggctgctccacctcgctctgattaagtactaatgaagtaattaaccgcgctaattaataataaatcgcacctacgtatgcacatgtggactcgcttgactaattaaatactgccatgcgaatgcgattagtggattatgaaaagaggaaatgtaagaactcatggctctctctgtgccatgcctgtactgcattgaaatgaaaactgatatacaa

SEQ ID NO: 2 - DNA, 1262 bp, Oryza sativa
gagctcaccggcgaggcggtgcgtctcctcggagatgtggtagaagctggcgccccatttcatggcggcgagcgggccgggcggcctcctgccgcagcgggattcggtggctagaccgatctgtgggtggaggacggggacgaggtagtggagacagaggcggcattggaagaggggaagaggaggaggaagtggtggcaggcagaggcggatgaggaacttgcgccagcgacgtggatatggagggggcgacggcaatggggaggcggcgatggaagcgaggagatgggcaggcggcggaggcagcggtggattttttttttctttttctttttcggaccctttcacctgctcggtgattcttcttttttatacagcacgacggcttctcctattcacgacgcctcggctggaccatggaccgttggccactggagcattcttccatgatctagattttttttttcactcaactttactacttcacatctgatggctggtgttgaattcattgtgcatccaacggtcattattaaattgatgacgtggcgcaatgaggtgacgaaacactttactttttttactactttagatctgtcggcaggagtcccagatatgtatacttgagctggattagttgggttttggatggagtaactttctgcagactgcaacattctgacacacgtagcagcacaaaagagttgcgaacaaacttggactgttaacatgtcaacgcataaaactgaaaaaaaaaacctgtcaaaatgcataataaataaaactgaaaaaaaataagaataaatgttgagagtgggatttgaacccacgccctttcggaccagaaccttaatctggcgccttagaccaactcggccatctcaactttttgctctgtcatccaaacaaagttataagaaatcatataataataactaagacttgatgcctcagtagtttagttaaactaatttgaatttgttagtacagtttgcatttcaaattgttccaatttggacgccacggctggtttcagttgctcacgacgcctcacacacatattttgcttccttgcttgtgacactagggcacaaaactccaacactcaaacgacacttcacgcatctctcctgaaatcttgcaccccccaactctgcatcgtcgcgtataaaatgcagaccaaaccccagctcaactctgcatcatcatcatcaactcgatagaaaaagaaagaaattaaaaagaaaatcacggcgcgtgaa

SEQ ID NO: 3 - DNA, 21 bp, Artificial Sequence
  Description of Artificial Sequence Synthetic primer
cacaggaaacaggatccgcgg

SEQ ID NO: 4 - DNA, 29 bp, Artificial Sequence
  Description of Artificial Sequence Synthetic primer
cgcgagctcgccggaaagtgaaattgacc

SEQ ID NO: 5 - DNA, 29 bp, Artificial Sequence
  Description of Artificial Sequence Synthetic primer
tactatacaactaaagccagctaaagatg

SEQ ID NO: 6 - DNA, 26 bp, Artificial Sequence
  Description of Artificial Sequence Synthetic primer
agccttgtgtgtgggcaacagtacca